(19) United States
(12) Patent Application Publication
(10) Pub. No.: US 2026/0207656 A1
(43) Pub. Date: Jul. 23, 2026

(54) COMPOSITION FOR PREVENTING AND/OR TREATING METABOLIC DISORDERS AND METHOD OF ITS PREPARATION

(71) Applicants: Pharmako Biotechnologies Pty Ltd, Frenchs Forest (AU); University of South Australia, Adelaide (AU)

(72) Inventors: George Kokkinis, Frenchs Forest (AU); Paul Matthew Joyce, Adelaide (AU); Clive Allan Prestidge, Adelaide (AU)

(21) Appl. No.: 19/140,174

(22) PCT Filed: Dec. 20, 2023
(86) PCT No.: PCT/AU2023/051335

§ 371 Date: Jun. 17, 2025

(30) Foreign Application Priority Data
Dec. 22, 2022 (AU) 2022903957

(51) Int. Cl. A61K 31/733; A61K 9/08; A61K 33/08; A61K 45/06; A61P 1/00; A61P 3/04; A61P 3/08

(52) U.S. Cl. CPC A61K 31/733; A61K 9/08; A61K 33/08; A61K 45/06; A61P 1/00; A61P 3/04; A61P 3/08

(57) ABSTRACT

The present disclosure relates to composition and methods for the treatment and prevention of metabolic diseases and disorders. According to a first aspect of the present invention, there is provided a composition for administration to a subject in need thereof comprising 1-99% (v/v) of an aqueous solution of at least 1% (w/v) inulin and/or one or more analogues thereof, and 1-99% (v/v) of a dispersed suspension of at least 1% (w/v) clay.

### TECHNICAL FIELD

The present disclosure relates to composition and methods for the treatment and prevention of metabolic diseases and disorders.

### BACKGROUND

Any references to methods, apparatus or documents of the prior art are not to be taken as constituting any evidence or admission that they formed, or form part of the common general knowledge.

With increasingly sedentary lifestyles and advanced technologies, obesity in humans has become one of the serious concerns. Obesity defines a preventable disease generally driven by intake-imbalanced, energy-rich diet high in fat and sugar, and lack of physical activity. Prevalence of obesity has increased markedly worldwide, doubling since 1980 as reported by the WHO in 2017.

Obesity, a metabolic disorder, has an effect on fat and/or lipid absorption in bloodstream, and can trigger other disorders in the body. For instance, obesity can lead to an accumulation of fats and sugars in the bloodstream, affecting the pancreas thereby leading to an insulin imbalance and potentially causing type-2 diabetes. In another instance, the accumulation of fats or lipids may affect liver function and cholesterol levels, leading to cardiovascular diseases. The rate and extent of fat digestion in the bloodstream is controlled by the ability of the digestive enzymes, specifically gastric and pancreatic lipases, to hydrolyse exogenous lipids into absorbable free fatty acids and monoglycerides. Such digestive enzymes contribute to the gut microbiome and to maintaining a healthy pH of the stomach and intestines.

In recent times, authorities have tried to increase awareness about the drastic consequences associated with obesity. Health initiatives and programs have regulated various attributes such as the fat content in the food, improvement in food labelling, elimination of trans fatty acids, taxations on energy dense foods that are high in fat and so on. However, such initiatives have proven to be largely ineffective and have failed to provide the expected results. Moreover, while advanced cosmetic procedures and surgical treatments like bariatric surgery, Cryolipolysis, CoolSculpting have increased in popularity, these expedited solutions risk complications, higher cost, and discomfort to the patients undergoing such treatments. They are also not widely available to all those in need.

There are supplements and appetite suppressants currently available (i.e., Lorcaserin, Sibutramine, orlistat etc) which can reduce appetite and/or weight by influencing how (or if) fat is absorbed into the bloodstream. However, a large number of these products have been withdrawn from the market in various jurisdictions due to adverse side effects. For example, Lorcaserin was withdrawn by the FDA after reports emerged of a link with cancer, while Sibutramine was implicated in cases of stroke and myocardial infarction.

One of the few lipase inhibitors currently widely available commercially is orlistat, a reverse inhibitor that attaches to the serine residue of both gastric and pancreatic lipases' active site. However, the orlistat inhibitor has slow effects on reducing obesity and prolonged consumption of such lipase inhibitors can lead to adverse and unpleasant side effects such as GI distress and diarrhoea. As cited in Aronne, L. J.; Powell, A. G.; Apovian, C. M. (2011), entitled “Emerging pharmacotherapy for obesity”. Expert Opinion on Emerging Drugs. 16 (3): 587-96, 13, cases of liver damage have been reported by the patients consuming orlistat. As the gut microbiome plays a vital role in lipid absorption activity, strengthening gut microbiome in addition to minimising the absorption of the lipids can assist in accelerating a healthy rate of weight loss.

Recently, research efforts have begun focusing on developing solutions for metabolic disorders which are natural and have reduced side effects, while being active in lowering fat consumption and/or absorption. For example, US patent publication U.S. Pat. No. 10,695,294B2 discloses a porous silica material for use as an active dietary ingredient, and is orally administered to subjects for lowering fat absorption.

European patent publication EP2879713B1 discloses a propionate inulin ester. The ester is known for use in therapy for the reduction of appetite, food intake and/or calorie intake. The ester can improve insulin sensitivity in a subject and can treat or prevent obesity or diabetes.

Another US Patent publication US20170027996A1 discloses a prebiotics composition including inulin, effects thereof to treat metabolic disorder. The composition, when administered to a diet-induced mouse, results in a loss of at least about 5% of the total body weight in just 10 days of regular administration. Canadian patent publication CA2777941 discloses a nutritional composition including a fructo-oligosaccharide (FOS) and a polysaccharide, which promotes gut microbiota balance and health.

However, these existing natural compositions are still slow to elicit a therapeutic benefit, and tend to be inefficient at reducing body weight and/or absorbing fat/lipids and preventing their entry into the bloodstream, and/or maintaining the health of the gut microbiome, which are root causes of obesity and other related disorders.

It would be attractive, practically and commercially, to develop natural based compositions, which have minimal adverse effects on the human body upon administration, and have ability not just to prevent lipids from entering the bloodstream (i.e., by adsorbing lipids from the gastrointestinal (GI) tract), and/or prevent the absorption of lipids into the bloodstream, but also to improve/maintain the health of the gut microbiome.

It is an object of the present invention to overcome or ameliorate the above discussed disadvantages of the prior art, or at least offer a useful alternative.

### SUMMARY

To overcome limitations in the prior art described above, and to overcome other limitations that will be apparent upon reading and understanding the present specification, aspects described herein are directed towards compositions, and methods for treating and preventing metabolic disorders.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, provided herein is a composition for administration to a subject in need thereof, comprising: 1-99% (v/v) of an aqueous solution of at least 1% (w/v) inulin and/or one or more analogues thereof, and 1-99% (v/v) of a dispersed suspension of at least 1% (w/v) clay.

In one embodiment, provided herein is a composition comprising about 30% (v/v) to about 80% (v/v) of an aqueous solution of about 1% (w/v) to about 5% (w/v) inulin and/or one or more analogues thereof, and about 30% (v/v) to about 50% (v/v) of a dispersed suspension of about 1% (w/v) to about 5% (w/v) clay.

In one aspect, provided herein is a method for preparing a composition as described herein, the method comprising the steps of:
preparing the aqueous solution by substantially dissolving the inulin molecules or analogues thereof in a first aqueous media;
preparing the dispersed suspension by dispersing the clay in a second aqueous media;
mixing the first aqueous solution with the second aqueous solution, to form a hybrid composition; and
optionally drying the hybrid composition to produce a dried composition of the invention.

In one embodiment, the hybrid composition comprises about 1% (w/v) to about 2.5% (w/v) inulin and/or one or more analogues thereof, and about 1% (w/v) to about 2.5% (w/v) clay.

In one embodiment, the method comprises the step of drying the hybrid composition by spray drying.

In one embodiment, the dried hybrid composition comprises about 30% (w/w) to about 80% (w/w) inulin and/or one or more analogues thereof, and about 20% (w/w) to about 50% (w/w) clay.

In one aspect, provided herein is a method for reducing the absorption of lipids into the bloodstream of a subject in need thereof, the method comprising the step of administering a therapeutically effective composition to said subject, wherein the composition comprises 1-99% (v/v) of an aqueous solution of at least 1% (w/v) inulin and/or one or more analogues thereof, and 1-99% (v/v) of a dispersed suspension of at least 1% (w/v) clay.

In one aspect, provided herein is a method for treating or preventing one or more metabolic disorders in a subject in need thereof, the method comprising the step of administering a therapeutically effective composition to said subject, wherein the composition comprises 1-99% (v/v) of an aqueous solution of at least 1% (w/v) inulin and/or one or more analogues thereof, and 1-99% (v/v) of a dispersed suspension of at least 1% (w/v) clay.

In one aspect, provided herein is a method for maintaining a weight, inducing weight loss, and/or maintaining blood glucose concentrations in a subject suffering from one or more metabolic disorders, the method comprising the step of administering a therapeutically effective composition to said subject, wherein the composition comprises 1-99% (v/v) of an aqueous solution of at least 1% (w/v) inulin and/or one or more analogues thereof, and 1-99% (v/v) of a dispersed suspension of at least 1% (w/v) clay.

In one embodiment, said composition is subjected to a spray-drying step and wherein the dried composition comprises about 30% (w/w) to about 80% (w/w) inulin and/or one or more analogues thereof, and about 20% (w/w) to about 50% (w/w) clay.

In one embodiment, said inulin molecules or analogues thereof have a degree of polymerization of between 2 and 60.

In one embodiment, said clay comprises or consists of montmorillonite.

In one embodiment, said clay comprises or consists of bentonite.

In one embodiment, the composition comprises an analogue of inulin that is oligofructose.

In one embodiment, the first and/or second aqueous media are selected from water, purified water, biological buffers, saline buffers, and/or pharmaceutical- or food-grade media and buffers.

In one embodiment, the composition comprises about 30% (v/v) to about 80% (v/v) of an aqueous solution at about 1% (w/v) to about 5% (w/v) of an analogue of inulin, and about 30% (v/v) to about 70% (v/v) of a dispersed suspension of about 1% (w/v) to about 5% (w/v) clay.

In one embodiment, the composition is a hybrid composition comprised of inulin and/or one or more analogues thereof, and clay.

In one embodiment, the composition comprises about 30% (v/v) to about 80% (v/v) of an aqueous solution at about 1% (w/v) to about 5% (w/v) of an analogue of oligofructose, and about 30% (v/v) to about 70% (v/v) of a dispersed suspension of about 1% (w/v) to about 5% (w/v) clay, wherein the clay is bentonite or montmorillonite or a mixture thereof.

In one embodiment, the composition is a hybrid composition.

In one embodiment, the composition comprises about 70% (w/w) inulin and/or one or more analogues thereof, and about 30% clay.

In one embodiment, the composition comprises about 50% (w/w) inulin and/or one or more analogues thereof, and about 50% clay.

In one embodiment, the composition is in a unit dosage form which comprises about 1 g to about 50 g of inulin and/or one or more analogues thereof.

In one embodiment, the composition is in a unit dosage form which comprises about 3.5 g of inulin and/or one or more analogues thereof.

In one embodiment, the composition is in a unit dosage form which comprises about 1 g to about 10 g of clay.

In one embodiment, the composition is in a unit dosage form which comprises about 1.5 g of clay.

In one embodiment, the composition is a dry composition.

In one embodiment, the composition further comprises a pharmaceutically acceptable excipient.

In one embodiment, the composition is pharmaceutically acceptable.

In one embodiment, the composition is formulated for oral administration.

In one embodiment, the composition is a stable composition.

In one embodiment, said metabolic disorder is selected from the group consisting of: obesity; irritable bowel syndrome (IBS); inflammatory bowel disease (IBD); diabetes mellitus; liver disease; familial hypercholesterolemia; elevated cholesterol; Gaucher disease; Hunter syndrome; Krabbe disease; atherosclerosis; Maple syrup urine disease; hypothyroidism; Metachromatic leukodystrophy; Niemann-Pick; Phenylketonuria (PKU); Porphyria; Tay-Sachs disease; and Wilson's disease. Preferably, the metabolic disorder excludes stroke or Mitochondrial encephalopathy, lactic acidosis, stroke-like episodes (MELAS).

In one embodiment, said metabolic disorder is selected from the group consisting of: obesity; diabetes mellitus; liver disease; familial hypercholesterolemia; elevated cholesterol; Gaucher disease; Hunter syndrome; and Niemann-Pick.

In one embodiment, the composition is administered to the subject 1-3 times per day.

In one embodiment, the composition is administered to the subject for at least 1-15 weeks.

In one embodiment, the composition is administered to the subject for 4-12 weeks.

In one embodiment, the composition is administered orally.

In one embodiment, the composition is administered in the fed state.

In one embodiment, the composition is administered in the fasted state.

One embodiment includes the use of inulin and/or one or more analogues thereof and clay in the manufacture of a medicament for the treatment of a metabolic disorder in a subject in need thereof.

Other embodiments of the invention will be evident from the following detailed description of various aspects of the invention.

### BRIEF DESCRIPTION OF THE DRAWINGS

The illustrated embodiments of the disclosed subject matter will be best understood by reference to the drawings, wherein like parts are designated by like numerals throughout. The following description is intended only by way of example, and simply illustrates certain selected embodiments of devices, systems, and processes that are consistent with the disclosed subject matter as claimed herein.

FIG. 1 is a schematic diagram illustrating an exemplary flowchart depicting a method for preparing a composition in accordance with an embodiment of the present invention.

FIG. 2 illustrates a scanned electron micrograph of a hybrid composition, in accordance with the embodiment of the present invention.

FIG. 3 illustrates a statistical analysis of experimental studies indicating a mean change in rodent body weight, normalized as a function of original body weight, in accordance with the embodiment of the present invention.

FIG. 4 illustrates a comparative analysis of the normalized change in rodent body weight after treatment for 3 weeks, in accordance with the embodiment of the present invention. Statistical significance was calculated using a one-way ANOVA followed by Tukey's post-test for multiple comparisons using GraphPad Prism Version 8.0 (GraphPad Software Inc., California), where **** represents p<0.0001.

FIG. 5 illustrates a comparative analysis of blood glucose concentrations in rodents following treatment for 3 weeks, in accordance with the embodiment of the present invention. Statistical significance was calculated using a one-way ANOVA followed by Tukey's post-test for multiple comparisons using GraphPad Prism Version 8.0 (GraphPad Software Inc., California), where * refers to p<0.05, and ** refers to p<0.01.

FIG. 6 illustrates a comparative analysis of Mean rodent liver weight, normalized as a function of overall body weight of the rodent, at the completion of the treatment period, in accordance with the embodiment of the present invention.

FIGS. 7A, 7B, and 7C illustrate comparative analysis of plasma concentrations of key hepatic enzymes: Lactate Dehydrogenase (LDH); Alanine aminotransferase (ALT), and Aspartate aminotransferase (AST) respectively at the completion of the treatment period, in accordance with the embodiment of the present invention. Statistical significance was calculated using a one-way ANOVA followed by Tukey's post-test for multiple comparisons using GraphPad Prism Version 8.0 (GraphPad Software Inc., California), where * refers to p<0.05, ** refers to p<0.01, and where **** represents p<0.0001.

FIG. 8A illustrates microbiome population profiles for subject animals at the Class taxonomical level, after a 3-week treatment period; and FIG. 8B illustrates principal coordinate analysis demonstrating the separation and beta diversity of the microbiome analysis for each treatment group respectively, in accordance with the embodiment of the present invention. In (i), principal coordinate 1 (x axis, PCo1) is plotted against principal coordinate 2 (y axis, PCo2). In (ii), PCo1 is plotted against principal coordinate 3 (PCo3) and in (iii) PCo2 is plotted against PCo3.

FIGS. 9A, 9B, 9C, 9D, 9E, and 9F illustrate relative abundance of key microbial communities at the phylum, family and genus taxonomical level following 3 weeks of treatment, in accordance with the embodiment of the present invention. Statistical significance was calculated using a one-way ANOVA followed by Tukey's post-test for multiple comparisons using GraphPad Prism Version 8.0 (GraphPad Software Inc., California), where * refers to p<0.05.

FIG. 10 in vitro lipid hydrolysis profiles under simulated fasted-state intestinal conditions (A), with corresponding area-under-the-curve (AUC) (B), extent of digestion (C), and degree of lipolysis inhibition (D) data. Hybrid microparticles comprised of inulin and montmorillonite inhibit in vitro lipid hydrolysis under simulated fasted-state intestinal conditions to a greater degree than the precursor materials and a physical mix of inulin and montmorillonite. Statistical significance was calculated using a one-way ANOVA followed by Tukey's post-test for multiple comparisons using GraphPad Prism Version 8.0 (GraphPad Software Inc., California), where ** refers to p<0.01, and where **** represents p<0.0001.

FIG. 11 In vitro lipid hydrolysis profiles under simulated fed-state intestinal conditions (A), with corresponding area-under-the-curve (AUC) (B), extent of digestion (C), and degree of lipolysis inhibition (D) data. Hybrid microparticles comprised of inulin and montmorillonite inhibit in vitro lipid hydrolysis under simulated fed-state intestinal conditions to a greater degree than the precursor materials and a physical mix of inulin and montmorillonite. Statistical significance was calculated using a one-way ANOVA followed by Tukey's post-test for multiple comparisons using GraphPad Prism Version 8.0 (GraphPad Software Inc., California), where ** refers to p<0.01, and where **** represents p<0.0001.

FIG. 12 Inulin, bentonite and INU-BEN (50:50 ratio) promote the in vitro growth of Blautia following 24 h culturing.

FIG. 13 Normalized change in body weight (A) (relative to the starting body weights) and corresponding AUC values (B) for the various interventions. Statistical significance was calculated using a one-way ANOVA followed by Tukey's post-test for multiple comparisons using GraphPad Prism Version 8.0 (GraphPad Software Inc., California), where **** represents p<0.0001.

FIG. 14 Mean blood glucose levels (A) and HbA1c levels (B) following 21 days of treatment while exposed to a high fat diet (HFD).

FIG. 15 Mean epididymal fat pad weight following 21 days of treatment while exposed to a high fat diet (HFD).

FIG. 16 Mean plasma TNF-α concentrations following 21 days of treatment while exposed to a high fat diet (HFD).

### DETAILED DESCRIPTION

Preferred features, embodiments and variations of the invention may be discerned from the following detailed description, which provides sufficient information for those skilled in the art to perform the invention. The detailed description is not to be regarded as limiting the scope of the preceding summary of the invention in any way.

The present invention is not limited to particular methods, and experimental conditions described, as such methods and conditions may vary. It is also to be understood that the terminology used herein is for the purpose of describing particular embodiments only, and is not intended to be limiting unless indicated, since the scope of the present invention will be limited only by the appended claims.

Unless stated otherwise, all technical and scientific terms and phrases used herein have the same meaning as commonly understood by one of ordinary skill in the art to which the invention belongs. Although any methods and materials similar or equivalent to those described herein can be used in the practice or testing of the present invention, the preferred methods and materials are now described.

### Definitions

Unless the context clearly requires otherwise, throughout the description and the claims, the words “comprise”, “comprising” and the like are to be construed in an inclusive sense as opposed to an exclusive or exhaustive sense; that is to say, in the sense of “including, but not limited to”.

Throughout the specification and claims (if present), unless the context requires otherwise, the term “substantially” or “about” will be understood to not be limited to the value for the range qualified by the terms.

The terms “a,” “an,” “the” and similar referents used in the context of describing the invention (especially in the context of the following claims) are to be construed to cover both the singular and the plural, unless otherwise indicated herein or clearly contradicted by context.

The use of any and all examples, or exemplary language (e.g., “such as”) provided herein is intended merely to better illuminate the invention and does not pose a limitation on the scope of the invention otherwise claimed. No language in the specification should be construed as indicating any non-claimed element is essential to the practice of the invention, unless otherwise indicated herein or clearly contradicted by context.

The term “treatment”, and the like, in the context of the compositions, dietary supplements, medicaments and methods of the present invention includes the alleviation of the symptoms related to a disease or disorder or an undesirable health condition that may reduce the life expectancy or quality of life of a subject. The treatment may cure the disease or disorder or improve the life expectancy or quality of life of the subject. Hence, in the context of this invention the word “treatment” or derivations thereof when used in relation to a therapeutic application includes all aspects of a therapy, such as the alleviation of pain associated with the disease or disorder, alleviation of the severity of the disease or disorder, improvement in one or more symptoms of the disease or disorder, improvement in the overall well-being of the subject being treated. Use of the word “treatment” or derivatives thereof will be understood to mean that the subject being “treated” may experience any one or more of the aforementioned benefits.

The term “prevention”, and the like, in the context of compositions, dietary supplements, medicaments and methods of the present invention refers to the prevention of the recurrence of all or some of the symptoms associated with a disease or disorder or an undesirable health condition that may reduce the life expectancy or quality of life of a subject, as well as the prevention of a disease or disorder or an undesirable health condition that may reduce the life expectancy or quality of life of a subject. The prevention may prevent morbidity, or delay morbidity due to a disease or disorder or an undesirable health condition that may reduce the life expectancy or quality of life of a subject. In this regard, it would be understood that the compositions, dietary supplements, medicaments and methods of the present invention may also be suitable for healthy subjects that have a desire to prevent the onset of a disease or disorder or an undesirable health condition that may reduce the life expectancy or quality of life of said subject.

The terms “therapeutically effective amount” or “therapeutic amount” are intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

In the context of this specification, the term “about” will be understood as indicating the usual tolerances that a skilled addressee would associate with the given value.

In the context of this specification, where a range is stated for a parameter it will be understood that the parameter includes all values within the stated range, inclusive of the stated endpoints of the range. Recitation of ranges of values herein is merely intended to serve as a shorthand method of referring individually to each separate value falling within the range. Unless otherwise indicated herein, each individual value is incorporated into the specification as if it were individually recited herein. For example, if a range is from about 1 to about 50, it is deemed to include, for example, 1, 7, 34, 46.1, 23.7, or any other value or range within the range.

The term “inulin” refers to a naturally occurring polysaccharides produced by many types of plants including agave, wheat, onion, bananas, garlic, asparagus, Jerusalem artichoke but is industrially most often extracted from chicory. Inulin is a heterogeneous collection of fructose polymers. It consists of chain-terminating glucosyl moieties and a repetitive fructosyl moiety, which are linked by B (2,1) bonds. The degree of polymerization (i.e. the number of monomer units in a polymer) of standard inulin ranges from 2 to 60. Inulins belong to a class of dietary fibers known as fructans, and are typically used by some plants as a means of storing energy in roots or rhizomes. Most plants that synthesize and store inulin do not store other forms of carbohydrate such as starch. Chicory root is the main source of extraction for commercial production of inulin. After harvest, the chicory roots are sliced and washed, then soaked in a solvent (hot water or ethanol); the inulin is then isolated, purified, and spray dried. Inulin may also be synthesized from sucrose.

The term “clay” as used herein may generally encompass fine-grained particles (the particles may also be referred to as “platelets” and these terms may be used interchangeably in the context of the clay dispersion) of earth material (comprising silicate), with the individual particles having a diameter of less than 5 microns. It would be understood that some clays may be prone to aggregate into aggregates that may be bigger than the 5 microns of the individual particles/platelets. The clay used in the present invention may be any material derived from the chemical and physical weathering of silicate containing rock, and therefore comprises silicate. The clay may comprise one or more silicate from one or more silicate groups selected from the following; ortho silicates (or nesosilicates), pyrosilicate (or sorosilicates), cyclic silicates (or ring silicates), chain silicates (or pyroxenes), double chain silicate (or amphiboles), sheet or phyllosilicates, 3-D (or tecto) silicates, and so on. Examples of clays that may be used in the present invention may comprise montmorillonite, halloysite, laponite, zeolite, beidellite, nontronite, hectorite, attapulgite, saponite, kaolinite. kaolin, alone or in combinations thereof.

As used herein, the term “dietary supplement” encompasses products that may be consumed (administered) orally that comprise substances that can supplement a diet to improve the health of a subject and/or address deficiencies in the subject's diet and/or compensate for a medical condition or prevent a medical condition from emerging. It would be understood the term may be used interchangeably with the term “nutritional supplement” and that dietary supplements may not be subject to same regulatory threshold as a pharmaceutical composition that is prescribed by a medical practitioner.

In the context of the present invention, the term “subject” refers to an animal, preferably a mammal, most preferably a human, who has experienced and/or exhibited at least one symptom associated with a metabolic disorder, or would like to prevent the onset of a metabolic disorder, with a cancer. The subject need not be an individual under the clinical care of a medical practitioner. The subject may be human or may be a non-human such that reference to a subject or individual means a human or a non-human, such as an individual of any species of social, economic or research importance including, but not limited to, members of the classifications of ovine, bovine, equine, porcine, feline, canine, primates, rodents, especially domesticated members of those classifications, such as sheep, cattle, horses and dogs. Further, as used herein, a “subject in need thereof” may additionally be a subject who has not exhibited any symptoms of a metabolic disorder, but who has been deemed by a physician, clinician or other medical profession to be at risk of developing a metabolic disorder. For example, the subject may be deemed at risk of developing a metabolic disorder (and therefore in need of prevention or preventive treatment) as a consequence of the subject's medical history, including, but not limited to, family history, pre-disposition, co-existing/contributory health issue.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and preferably together in the same formulation.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

The term “pharmaceutically acceptable” as used herein refers to substances that do not cause substantial adverse allergic or immunological reactions when administered to a subject. A “pharmaceutically acceptable carrier” includes, but is not limited to, solvents, coatings, dispersion agents, wetting agents, isotonic and absorption delaying agents and disintegrants.

“Delamination” is defined as the mechanical separation of interlaminar layers or plies and “delaminated” clay is the product of this process. Delamination causes large aggregates of clay minerals to be converted into high surface area clay platelets of smaller particle size.

The term “hybrid” in the context of the present invention refers to particles formed from inulin and clay. Further, “hybrid composition” refers to compositions comprising said hybrid particles. The characteristics of the hybrid composition can vary depending on the composition used and the drying method. Generally, in a hybrid, the inulin substantially encapsulate or coat the clay platelets, forming spherical micro- and nano-particles with diameters in the range of about 1 to about 10 μm (about 1000 to about 10,000 nm).

In the context of the present invention, the term “microbiome” relates to the microbial population that lives in the gastrointestinal (GI) tract of a subject, and the microbial population may include bacteria, viruses, fungi, protozoa, and so on. It is understood that many metabolic disorders can either create, or be attributed to, a lack of appropriate diversity in the gut microbiome, a lack of robustness of the microbial strains, or imbalances in the respective populations of beneficial and detrimental microbes in the subject.

### Description of the Preferred Embodiments

The present invention is predicated on the surprising finding that the combination of inulin (and/or one or more analogues thereof) and dispersed clay suspension can provide health and therapeutic benefits, particularly in comparison to the health and therapeutic benefits provided independently by inulin (and/or one or more analogues thereof) and a dispersed clay.

According to a first aspect of the present invention, there is provided a composition for administration to a subject in need thereof comprising 1-99% (v/v) of an aqueous solution of at least 1% (w/v) inulin and/or one or more analogues thereof, and 1-99% (v/v) of a dispersed suspension of at least 1% (w/v) clay.

In one embodiment, the composition comprises about 30% (v/v) to about 80% (v/v) of an aqueous solution of about 1% (w/v) to about 5% (w/v) inulin and/or one or more analogues thereof, and about 30% (v/v) to about 50% (v/v) of a dispersed suspension of about 1% (w/v) to about 5% (w/v) clay.

In one embodiment, the composition comprises about 30% (v/v) to about 80% (v/v), about 40% (v/v) to about 80% (v/v), about 50% (v/v) to about 80% (v/v), about 50% (v/v) to about 70% (v/v), about 60% (v/v) to about 70% (v/v), about 50% (v/v), about 60% (v/v) or about 70% (v/v) of an aqueous solution of about 1% (w/v) to about 5% (w/v), 1.5% (w/v) to about 5% (w/v), 2% (w/v) to about 5% (w/v), 2.5% (w/v) to about 5% (w/v), 3% (w/v) to about 5% (w/v), 3.5% (w/v) to about 5% (w/v), 4% (w/v) to about 5% (w/v) or 4.5% (w/v) to about 5% (w/v) inulin and/or one or more analogues thereof.

In one embodiment, the composition comprises about 30% (v/v) to about 50% (v/v), about 30% (v/v) to about 40% (v/v), about 35% (v/v) to about 40% (v/v), about 40% (v/v) to about 50% (v/v) or about 45% (v/v) to about 50% (v/v) of a dispersed suspension of about 1% (w/v) to about 5% (w/v), about 1.5% (w/v) to about 5% (w/v), about 2% (w/v) to about 5% (w/v), about 2.5% (w/v) to about 5% (w/v), about 3% (w/v) to about 5% (w/v), about 3.5% (w/v) to about 5% (w/v), about 4% (w/v) to about 5% (w/v), about 3%, about 3.5%, about 4%, or about 4.5% clay.

The inulin may be a heterogenous or homogenous preparation of any one or more inulin molecules selected from the group of polydisperse β(2-1) fructans (or polydisperse carbohydrate material predominantly comprising β(2-1) fructosyl-fructose links) present as plant storage carbohydrates. The inulin molecules would generally have a degree of polymerisation (DP) of 2-60, and would encompass inulin molecules currently known, as well as inulin molecules yet to identified and/or characterised. Without wishing to be limited by theory, it is postulated the β(2-1) bonds between the fructose molecules of the inulin prevents the inulin from being digested like a typical carbohydrate, thereby reducing the caloric intake associated with consuming inulin, whilst also providing dietary fibre benefits.

The inulin analogue may be any closely related to fructan comprising the β(2-1) bonds between the fructose molecules, with a DP of 2-60, which provides the same nutritional benefits as inulin. Preferably, in embodiments of the invention, the inulin analogue is one or more oligofructose molecule, or polyfructose molecule (i.e. sinistrin), which may also be derived from plants. The inulin and analogues thereof may be naturally derived from a plant or may be a synthetic or modified version of a naturally occurring inulin, and the aqueous solution in the composition of the invention may comprise only inulin, or only one or more analogues of inulin, or may comprise inulin and one or more analogues thereof. Preferably, the inulin or analogues thereof used in the compositions of the present invention are food-grade or pharmaceutical grade materials.

The inulin and analogues thereof may have a DP of 2-60, indicating the molecules may be 2-60 units long. However, it would be understood that preparations of inulin or analogues thereof would generally be heterogenous, in that they would comprise individual polymers with lengths ranging from 2-60 monomer unit, but the average DP maybe any value between 2-60. For example, the average DP for inulin extracted from sugar beet roots is 10-12, while the average DP for inulin extracted from chicory is 10-20, but both inulins have a distribution of molecules with chain lengths generally between 2 and 60 (i.e. a DP of 2-60). On the other hand, the average DP for oligofructose is 4-6, and oligofructose has a distribution of molecules with chain lengths generally between 2 and 10 (i.e. a DP of 2-10).

Accordingly, in embodiments of the invention, the compositions may comprise an aqueous solution of at least 1% (w/v) inulin and/or one or more analogues thereof, wherein the average DP of the inulin or analogue thereof in between 2 and 50, or between 2 and 40, or between 2 and 30, or between 2 and 20, or between 2 and 10, or between 10 and 20, or between 15 and 45, or between 5 and 25, or between 5 and 15.

The clay used in the compositions and methods of the present invention would be suitable for the required administration routes, and would preferably be food-grade or pharmaceutical-grade clays. In preferred embodiments of the invention, the clay may comprise at least 50%, or at least 60%, or at least 70%, or at least 80% or at least 90% montmorillonite. In particular preferred embodiments of the invention, the clay is bentonite, which predominantly comprises montmorillonite.

The dispersed suspension may be prepared by substantially dispersing the clay in any suitable aqueous media, including, but not limited to, water, purified water, biological buffers, saline buffers, and/or pharmaceutical- or food-grade media and buffers. Preferably, the clay platelets in the dispersed suspension comprising at least 1% (w/v) clay are substantially delaminated. The dispersion may be achieved by simple mixing, blending, stirring, agitation, sonication, and/or combinations thereof.

In a further aspect of the present invention, there is provided a method for preparing a composition according to the invention, comprising the steps of preparing the aqueous solution by dissolving the inulin molecules or analogues thereof in a first aqueous media, preparing the dispersed solution by dispersing the clay in a second aqueous media, mixing the first aqueous solution with the second aqueous solution, to form a hybrid composition, and optionally drying the hybrid composition to produce the composition of the invention.

In one aspect, provided herein is a method for preparing a composition as described herein, the method comprising the steps of:
preparing the aqueous solution by substantially dissolving the inulin molecules or analogues thereof in a first aqueous media;
preparing the dispersed suspension by dispersing the clay in a second aqueous media;
mixing the first aqueous solution with the second aqueous solution, to form a hybrid composition; and
optionally drying the hybrid composition to produce a dried composition of the invention.

The aqueous solution may be prepared by substantially dissolving the inulin and or analogues thereof in any suitable aqueous media, including, but not limited to, water, purified water, biological buffers, saline buffers, and/or pharmaceutical- or food-grade media and buffers. In embodiments of the invention wherein the inulin or analogues thereof have a longer average DP, the aqueous solution may be incompletely dissolved and/or may required heating to completely dissolve the inulin or analogues thereof in the aqueous media.

The preparation of the second aqueous media may preferably involve rapid stirring for at least 0.5, at least 1 or at least 2.5 hours at room temperature, or until clay platelets formed undergo delaminating. Thereafter, the method involves mixing the first aqueous solution with the second aqueous solution, forming a hybrid composition. Finally, the hybrid composition undergoes drying, wherein said drying is performed by a means selected from the group consisting of spray drying, freeze drying, convective drying, contact drying, dielectric drying, supercritical drying, or combinations thereof. The hybrid composition preferably undergoes spray drying.

When used, the drying may be performed by any suitable means, including, but not limited to, spray drying, freeze drying, convective drying, contact drying, dielectric drying, supercritical drying, and/or combinations thereof. In preferred embodiments of the method, the hybrid composition undergoes drying by the method of spray drying.

In embodiments of the composition, the composition further comprised one or more excipients and/or one or more additional active components. In the context of the present invention, the excipient is any excipient that is biocompatible in the context of the relevant administration method, i.e., the material may be incorporated into a composition of the present invention and administered to a subject without causing any undesirable or undue biological effects, including but not limited to undesirable or undue toxicity, incompatibility, instability, irritation, allergic response and the like. In a preferred embodiment, the excipient is approved or approvable by a regulatory agency or body for use in subjects.

Said excipients and/or active components may be added to the composition by any means suitable, and may be added as a solid or aqueous component, and may be added when the composition or components thereof are in liquid or solid form. For example, excipient(s) and/or active component(s) may be added to the aqueous solution or the dispersed suspension separately, or may be added to the composition after the aqueous solution are mixed, or may be added to the composition after the composition is dried. In other examples, the excipient(s) and/or active component(s) are added or present in the starting (dry) preparation of the inulin or analogues there and/or clay prior to preparing the aqueous solution and dispersed suspension, respectively, or may be present in the aqueous media used to prepared the aqueous solution and/or dispersed suspension.

In embodiments of the invention, the excipients or additional active component may be any compound, reagent or component selected from the group consisting of pharmaceutical formulations, nutraceuticals, supplements, vitamins, minerals, antioxidants, fish oils, DHA, EPA, omega-rich fats, protein, fats, probiotics, prebiotics, hormones, phytonutrients, nanoparticles, preservatives, colour modifiers, paste modifiers, taste modifiers, acidity regulators, fillers, binders, glidants, disintegrants, lubricants, coatings, and/or combinations thereof. The additional compound, component or reagent may also be an active compound, reagent or component known to be useful in the treatment or prevention of metabolic disorders, such as, but not limited to orlistat, and/or compounds, reagents or components useful for maintaining gut health, such as, but not limited to, selenium.

In embodiments of the present invention, the composition comprises at least 10% (v/v) of an aqueous solution of at least 1% (w/v) inulin and/or one or more analogues thereof, and at least 10% (v/v) of a dispersed suspension of at least 1% (w/v) clay. In other embodiments of the invention, the composition comprises at about 30% (v/v) to about 50% (v/v) of an aqueous solution of about 1% (w/v) to about 5% (w/v) inulin and/or one or more analogues thereof, and about 30% (v/v) to about 50% (v/v) of a dispersed suspension of about 1% (w/v) to about 5% (w/v) clay.

In one embodiment, the hybrid composition comprises about 1% (w/v) to about 5% (w/v), about 1.5% (w/v) to about 5% (w/v), about 2% (w/v) to about 5% (w/v), about 2.5% (w/v) to about 5% (w/v), about 3% (w/v) to about 5% (w/v), about 3.5% (w/v) to about 5% (w/v), about 4% (w/v) to about 5% (w/v), about 3%, about 3.5%, about 4%, or about 4.5% inulin and/or one or more analogues thereof.

In one embodiment, the hybrid composition comprises about 1% (w/v) to about 5% (w/v), about 1.5% (w/v) to about 5% (w/v), about 2% (w/v) to about 5% (w/v), about 2.5% (w/v) to about 5% (w/v), about 3% (w/v) to about 5% (w/v), about 3.5% (w/v) to about 5% (w/v), about 4% (w/v) to about 5% (w/v), about 3%, about 3.5%, about 4%, or about 4.5% clay.

In one embodiment, the hybrid composition comprises about 1% (w/v) to about 2.5% (w/v) inulin and/or one or more analogues thereof, and about 1% (w/v) to about 2.5% (w/v) clay.

In one embodiment, the method comprises the step of drying the hybrid composition by spray drying.

In one embodiment, the dried hybrid composition comprises about 30% (w/w) to about 80% (w/w) inulin and/or one or more analogues thereof, and about 20% (w/w) to about 50% (w/w) clay.

It would be understood, in particular embodiments, that the composition may comprise other components, solutions and/or dispersions besides the aqueous solution and the dispersed suspension, such that combined % (v/v) of the aqueous solution of at least 1% (w/v) inulin and/or one or more analogues thereof, and the % (v/v) of a dispersed suspension of at least 1% (w/v) clay in the composition does not equate to 100% (v/v). Alternatively, in embodiments, other components, solutions and/or dispersions, besides the aqueous solution and the dispersed suspension, may be excluded (e.g. such that the composition consists essentially of inulin and/or one or more analogues thereof, and the % (v/v) of a dispersed suspension of at least 1% (w/v) clay).

In other embodiments of the invention, the composition is a dried composition comprising about 30% (w/w) to about 50% (w/w) of inulin and/or one or more analogues thereof and about 30% (w/w) to about 50% (w/w) clay. In further embodiments of the invention, the composition is a dried composition comprising about 10% (w/w) to about 30% (w/w) of inulin and/or one or more analogues thereof and about 10% (w/w) to about 30% (w/w) clay. In these embodiments, the composition is dried after first combining the aqueous solution of at least 1% (w/v) inulin and/or one or more analogues thereof, and the dispersed suspension of at least 1% (w/v) clay as described herein, before drying. The final % (w/w) of the components of the dried composition would be determined by the starting % (w/v) of the aqueous solution of inulin and/or one or more analogues thereof, and the dispersed suspension of clay, as well at the % (v/v) of the aqueous solution and dispersed suspension used in the composition prior to drying.

For example, in embodiments of the invention wherein, prior to drying, the composition comprises 20% (v/v) of an aqueous solution of 5% (w/v) inulin and/or one or more analogues thereof, and 80% (v/v) of a dispersed suspension of 5% (w/v) clay, the resulting dried composition would comprise 20% (w/w) inulin and/or one or more analogues thereof, and 80% (w/w) clay. In another example, in embodiments of the invention wherein, prior to drying, composition comprises 20% (v/v) of an aqueous solution of 20% (w/v) inulin and/or one or more analogues thereof, and 80% (v/v) of a dispersed suspension of 5% (w/v) clay, the resulting dried composition would comprise 50% (w/w) inulin and/or one or more analogues thereof, and 50% (w/w) clay.

In various embodiments of the present invention, the composition may be provided in a form selected from at least one or more of powder, solid, liquid, viscous, vapours, inhalers, infusions, granules, compressed powder, tablet, pill, sachet, pellets, caplet, and/or combinations thereof.

In one embodiment, the composition is provided in powder or compressed powder form.

According to another aspect, there is provided the use of a composition of the present invention in the manufacture of a medicament for reducing the absorption of lipids into the bloodstream of a subject in need thereof.

According to another aspect, there is provided the use of a composition of the present invention in the manufacture of a dietary supplement reducing the absorption of lipids into the bloodstream of a subject in need thereof.

In one aspect, provided herein is a method for reducing the absorption of lipids into the bloodstream of a subject in need thereof, the method comprising the step of administering a therapeutically effective composition to said subject, wherein the composition comprises 1-99% (v/v) of an aqueous solution of at least 1% (w/v) inulin and/or one or more analogues thereof, and 1-99% (v/v) of a dispersed suspension of at least 1% (w/v) clay.

In one aspect, provided herein is a method for treating or preventing one or more metabolic disorders in a subject in need thereof, the method comprising the step of administering a therapeutically effective composition to said subject, wherein the composition comprises 1-99% (v/v) of an aqueous solution of at least 1% (w/v) inulin and/or one or more analogues thereof, and 1-99% (v/v) of a dispersed suspension of at least 1% (w/v) clay.

In one aspect, provided herein is a method for maintaining a weight, inducing weight loss, and/or maintaining blood glucose concentrations in a subject suffering from one or more metabolic disorders, the method comprising the step of administering a therapeutically effective composition to said subject, wherein the composition comprises 1-99% (v/v) of an aqueous solution of at least 1% (w/v) inulin and/or one or more analogues thereof, and 1-99% (v/v) of a dispersed suspension of at least 1% (w/v) clay.

In one embodiment, provided herein is a method of reducing the relative abundance of harmful microbial populations in the gut microbiota.

In one embodiment, said composition is subjected to a drying step and wherein the dried composition comprises about 30% (w/w) to about 80% (w/w) inulin and/or one or more analogues thereof, and about 20% (w/w) to about 50% (w/w) clay.

In one embodiment, said inulin molecules or analogues thereof have a degree of polymerization of between 2 and 60.

In one embodiment, said clay comprises or consists of montmorillonite.

In one embodiment, said clay comprises or consists of bentonite.

In one embodiment, the composition comprises an analogue of inulin that is oligofructose.

In one embodiment, the first and/or second aqueous media are selected from water, purified water, biological buffers, saline buffers, and/or pharmaceutical- or food-grade media and buffers.

In one embodiment, the composition comprises about 30% (v/v) to about 80% (v/v) of an aqueous solution at about 1% (w/v) to about 5% (w/v) of an analogue of inulin, and about 30% (v/v) to about 70% (v/v) of a dispersed suspension of about 1% (w/v) to about 5% (w/v) clay.

In one embodiment, the composition is a hybrid composition comprised of inulin and/or one or more analogues thereof, and clay.

In one embodiment, about 30% (v/v) to about 80% (v/v) of an aqueous solution at about 1% (w/v) to about 5% (w/v) of oligofructose, and about 30% (v/v) to about 70% (v/v) of a dispersed suspension of about 1% (w/v) to about 5% (w/v) bentonite or montmorillonite or a mixture thereof.

In one embodiment, the composition is a hybrid composition.

In one embodiment, the composition comprises about 70% (w/v) inulin and/or one or more analogues thereof, and about 30% (w/v) clay.

In one embodiment, the composition comprises about 50% (w/v) inulin and/or one or more analogues thereof, and about 50% (w/v) clay.

In one embodiment, the compositions of the present invention are in unit dosage form, such as tablets, capsules or the like. Such unit dosage forms may contain from about 5 mg to about 50 grams of inulin. In one example the unit dose contains about 5 mg to about 100 mg of inulin. In another example, the unit dose contains about 100 mg to about 500 mg of inulin. In another example, the unit dose contains about 500 mg to about 1000 mg of inulin. In another example, the unit dose contains about 1000 mg to about 2000 mg of inulin. In another example, the unit dose contains about 2000 mg to about 3000 mg of inulin. In another example, the unit dose contains about 3000 mg to about 4000 mg of inulin. In another example, the unit dose contains about 4000 mg to about 5000 mg of inulin. In another example, the unit dose contains about 1 g to about 2 g of inulin. In another example, the unit dose contains about 2 g to about 4 g of inulin. In another example, the unit dose contains about 4 g to about 8 g of inulin. In another example, the unit dose contains about 8 g to about 10 g of inulin. In another example, the unit dose contains about 10 g to about 15 g of inulin. In another example, the unit dose contains about 15 g to about 20 g of inulin. In another example, the unit dose contains about 20 g to about 30 g of inulin. In another example, the unit dose contains about 30 g to about 40 g of inulin. In another example, the unit dose contains about 40 g to about 50 g of inulin.

In another embodiment, such unit dosage forms may contain from about 5 mg to about 50 grams of clay. In one example the unit dose contains about 5 to 100 mg of clay. In another example, the unit dose contains about 100 to 500 mg of clay. In another example, the unit dose contains about 500 to about 1000 mg of clay. In another example, the unit dose contains about 1000 to about 2000 mg of clay. In another example, the unit dose contains about 2000 to about 3000 mg of clay. In another example, the unit dose contains about 3000 to about 4000 mg of clay. In another example, the unit dose contains about 4000 to about 5000 mg of clay. In another example, the unit dose contains about 1 g to about 2 g of clay. In another example, the unit dose contains about 2 g to about 4 g of clay. In another example, the unit dose contains about 4 g to about 8 g of clay. In another example, the unit dose contains about 8 g to about 10 g of clay. In another example, the unit dose contains about 10 g to about 15 g of clay. In another example, the unit dose contains about 15 g to about 20 g of clay. In another example, the unit dose contains about 20 g to about 30 g of clay. In another example, the unit dose contains about 30 g to about 40 g of clay. In another example, the unit dose contains about 40 g to about 50 g of clay.

In one embodiment, the composition is in a unit dosage form which comprises about 1 g to about 50 g of inulin and/or one or more analogues thereof.

In one embodiment, the composition is in a unit dosage form which comprises about 3.5 g of inulin and/or one or more analogues thereof.

In one embodiment, the composition is in a unit dosage form which comprises about 1 g to about 10 g of clay.

In one embodiment, the composition is in a unit dosage form which comprises about 1.5 g of clay.

In one embodiment, the composition is a dry composition.

In one embodiment, the composition further comprises a pharmaceutically acceptable excipient.

In one embodiment, the composition is pharmaceutically acceptable.

In one embodiment, the composition is formulated for oral administration.

In one embodiment, the composition is a stable composition.

Without wishing to be limited by theory, it is proposed that the compositions, medicaments and dietary supplements of the present invention may reduce the absorption of lipid through lipid adsorption onto the clay particles, thereby preventing their absorption into the bloodstream.

According to a further aspect, there is provided the use of a composition of the present invention in the manufacture of a medicament for treating or preventing metabolic disorders in a subject in need thereof.

According to a further aspect, there is provided the use of a composition of the present invention in the manufacture of a dietary supplement for treating or preventing metabolic disorders in a subject in need thereof.

According to yet another aspect, there is provided the use of a composition of the present invention in the manufacture of a medicament for maintaining a weight, inducing weight loss, and/or maintaining blood glucose concentrations in a subject suffering from one or more metabolic disorders.

According to yet another aspect, there is provided the use of a composition of the present invention in the manufacture of a dietary supplement for maintaining a weight, inducing weight loss, and/or maintaining blood glucose concentrations in a subject suffering from one or more metabolic disorders.

In one embodiment, said metabolic disorder is selected from the group consisting of: obesity; irritable bowel syndrome (IBS); inflammatory bowel disease (IBD); diabetes mellitus; liver disease; familial hypercholesterolemia; elevated cholesterol; Gaucher disease; Hunter syndrome; Krabbe disease; atherosclerosis; Maple syrup urine disease; hypothyroidism; Metachromatic leukodystrophy; Niemann-Pick; Phenylketonuria (PKU); Porphyria; Tay-Sachs disease; and Wilson's disease. Preferably, the metabolic disorder excludes stroke or Mitochondrial encephalopathy, lactic acidosis, stroke-like episodes (MELAS).

In one embodiment, said metabolic disorder is selected from the group consisting of: obesity; diabetes mellitus; liver disease; Familial hypercholesterolemia; elevated cholesterol; Gaucher disease; Hunter syndrome; and Niemann-Pick.

In particularly preferred embodiments of the invention, the metabolic disorder is selected from the group consisting of obesity, IBS/IBD, diabetes, liver disease and hypercholesterolemia.

It would be understood that a composition, medicament or dietary supplement that, when administered to a subject, is capable of reducing the absorption and transport of lipids into the bloodstream may have a beneficial effect on a subject. The lipids may encompass any dietary lipid, including the products of lipid digestion present in the GI tract.

Without wishing to be bound by theory, it is postulated that the therapeutic benefits of the compositions, dietary supplements and medicaments of the present invention may, in part, be attributed to the effects the compositions, dietary supplements and medicaments have on the subject's microbiome when administered.

Other and further aspects and features of the different embodiments of the present invention will be evident from reading the following detailed description of the embodiments, which are intended to illustrate, not limit, the present disclosure.

### Compositions and Medicaments, Dosages and Administration

The compositions, dietary supplements and medicaments of the present invention can be administered by oral, topical and/or parenteral routes. Preferred routes of administration include orally via capsules, tablets or powder that can be reconstituted into a consumable drink product.

Effective doses of the compositions, dietary supplements and medicaments used in the present invention may be ascertained by conventional methods. The specific dosage level required for any particular subject will depend on a number of factors, including the severity of the condition being treated, the route of administration and the weight of the subject.

The compositions, dietary supplements and medicaments may be administered once, twice, three, four or five times daily, or may be administered every second or third day, or once every week, once every two weeks or once every four weeks.

Some embodiments of the present invention may involve administration of the composition or medicament in multiple, separate doses. Accordingly, the methods of treatment and prevention described herein encompass the administration of multiple separated doses to a subject, for example, over a defined period of time.

In one embodiment, the composition is administered to the subject 1-3 times per day.

In one embodiment, the composition is administered to the subject for at least 1-15 weeks.

In one embodiment, the composition is administered to the subject for 4-12 weeks.

In one embodiment, the composition is administered orally.

In one embodiment, the composition is administered in the fed state.

In one embodiment, the composition is administered in the fasted state.

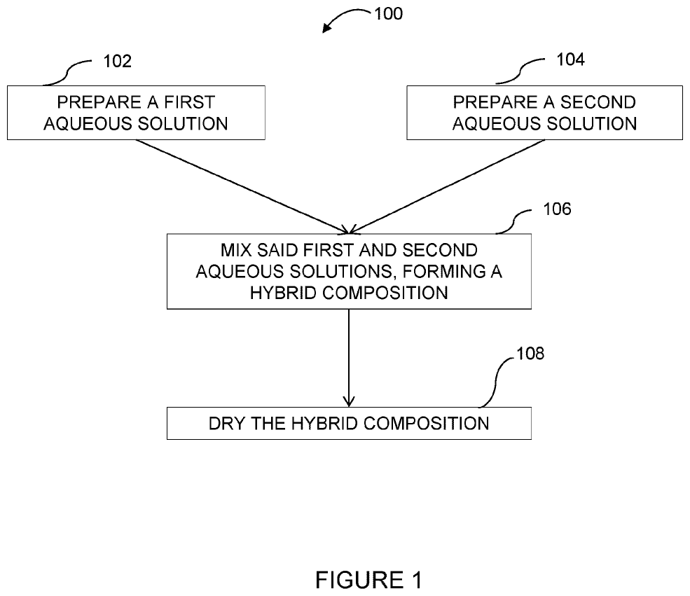

According to an aspect of the present invention, there is provided a method 100 for preparing the composition as illustrated in a flowchart in FIG. 1. The method 100 includes preparing an aqueous solution at step 102. The preparation of the aqueous solution includes dissolving the inulin molecules or analogues thereof in a first aqueous media. An example of the aqueous media may include, such as, but is not limited to, purified water. The inulin molecules or analogues thereof undergo stirring in the aqueous media for at least 30 minutes at room temperature.

Simultaneously, at step 104, a clay dispersed suspension is prepared. The dispersion is prepared by dispersing clay in a second aqueous media, forming a clay dispersion. The clay platelets in the dispersion are delaminated by stirring for at least 2 hours at room temperature.

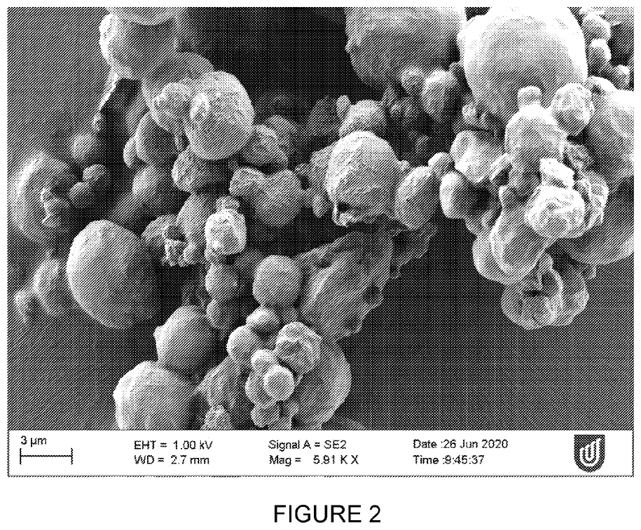

At step 106, the aqueous solution is mixed with the dispersed suspension to form a hybrid composition, and the hybrid composition undergoes a spray drying at step 108. An example of a hybrid composition of the invention was characterised and found to contain spherical hybrid particles of diameters in the range of 1-10 μm. The inulin particles are found to encapsulate or coat the clay platelets; however, the characteristics of the hybrid composition can vary depending on the composition used and the drying method. A scanning electron micrograph of the hybrid composition is shown in FIG. 2.

The hybrid composition is proposed to be useful for treating and preventing metabolic disorders related to lipid metabolism. Accordingly, experimental studies were conducted in vitro to test the ability of the hybrid compositions to inhibit lipid digestion.

Montmorillonite and bentonite are both types of clay minerals, but they have some differences in their composition and properties. Montmorillonite is a specific type of clay mineral that belongs to the smectite group, which also includes other clay minerals such as bentonite. Bentonite, on the other hand, is a broader term that refers to a group of clay minerals, of which montmorillonite is one of the key components. Montmorillonite and bentonite both have high water absorption capacity and can expand greatly when exposed to water and they have a high swelling ability. However, the overall performance of bentonite may vary depending on the specific composition and ratio of minerals present. Montmorillonite has a high cation exchange capacity, which refers to its ability to attract and hold positively charged ions (cations) within its structure. This property allows montmorillonite to bind and exchange various ions, which can be beneficial in applications such as soil conditioning. Bentonite, as a group, also exhibits significant cation exchange capacity.

Montmorillonite, a phyllosilicate, is a soft type of mineral that exists in small crystals which accumulate to form clay. Phyllosilicates or sheet silicates, are a group of minerals that include the mica, chlorite, serpentine, talc, and the clay minerals. Bentonite is an absorbent aluminium phyllosilicate clay consisting mostly of montmorillonite. In simpler terms, montmorillonite is a type of clay mineral found within bentonite.

In a further aspect, compositions of the present invention may be co-administered to a subject in need with one or more substances useful for the treatment or prevention of a metabolic disorder. In an embodiment, a composition of the invention may be co-administered with one or more compounds selected from the group consisting of sulfonylureas, non-sulfonylurea secretagogues, insulin, insulin analogues, glucagon-like peptides, exendin-4 polypeptides, beta 3 adrenoceptor agonists, PPAR agonists, dipeptidyl peptidase IV inhibitors, biguanides, alpha-glucosidase inhibitors, immunomodulators, statins and statin-containing combinations, angiotensin converting enzyme inhibitors, adeno sine A1 receptor agonists, adenosine A2 receptor agonists, aldosterone antagonists, alpha 1 adrenoceptor antagonists, alpha 2 adrenoceptor agonists, alpha 2 adrenoceptor agonists, angiotensin receptor antagonists, antioxidants, ATPase inhibitors, atrial peptide agonists, beta adrenoceptor antagonists, calcium channel agonists, calcium channel antagonists, diuretics, dopamine D1 receptor agonists, endopeptidase inhibitors, endothelin receptor antagonists, guanylate cyclase stimulants, phosphodiesterase V inhibitors, protein kinase inhibitors, Cdc2 kinase inhibitors, renin inhibitors, thromboxane synthase inhibitors, vasopeptidase inhibitors, vasopressin I antagonists, vasopressin 2 antagonists, angiogenesis inhibitors, advanced glycation end product inhibitors, bile acid binding agents, bile acid transport inhibitors, bone formation stimulants, apolipoprotein A1 agonists, DNA topoisomerase inhibitors, cholesterol absorption inhibitors, cholesterol antagonists, cholesteryl ester transfer protein antagonists, cytokine synthesis inhibitors, DNA polymerase inhibitors, dopamine D2 receptor agonists, endothelin receptor antagonists, growth hormone antagonists, insulin sensitizers, lipase inhibitors, lipid peroxidation inhibitors, lipoprotein A antagonists, microsomal transport protein inhibitors, microsomal triglyceride transfer protein inhibitors, nitric oxide synthase inhibitors, oxidizing agents, phospholipase A2 inhibitors, radical formation agonists, platelet aggregation antagonists, prostaglandin synthase stimulants, reverse cholesterol transport activators, rho kinase inhibitors, selective estrogen receptor modulators, squalene epoxidase inhibitors, squalene synthase inhibitors, thromboxane A2 antagonists, amylin agonists, cannabinoid receptor antagonists, cholecystokinin A agonists, corticotropin-releasing factor agonists, dopamine uptake inhibitors, G protein-coupled receptor modulators, glutamate antagonists, glucagon-like peptide-1 agonists, insulin sensitizers, melanin-concentrating hormone receptor antagonists, nerve growth factor agonists, neuropeptide Y agonists, neuropeptide Y antagonists, SNRIs, protein tyrosine phosphatase inhibitors, and serotonin 2C receptor agonists. In an embodiment, a composition of the present invention is co-administered with a lipase inhibitor (e.g. orlistat).

Further examples of the invention are described below. However, it should be noted that the invention should not be limited to these examples, and that the invention is susceptible to variations, modifications and/or additions other than those specifically described, and it is to be understood that the invention includes all such variations, modifications and/or additions which fall within the scope of the claims.

### EXAMPLES

### General Comments

Inulin/oligofructose (Frutafit® HD; Sensus) and purified bentonite (Veegum® HS; Vanderbilt Minerals, LLC) were used for all studies in this work.

### Example 1—Preparation of Inulin/Clay Composition (INU-BEN)

a) Inulin molecules (1 g; Frutafit® HD) were dissolved in purified water (50 ml) and stirred for at least 30 minutes at room temperature to produce an aqueous inulin solution.
b) Separately, a clay dispersed suspension was prepared by dispersing purified bentonite (Veegum® HS; 1 g) in 50 ml purified water and mixed (by hand with vigorous shaking for 1 min), forming a clay dispersed suspension. The clay platelets in the clay dispersed suspension were delaminated by stirring using a magnetic stirrer at 300 rpm for at least 2 hours at room temperature.
c) The aqueous inulin solution from (a) was then mixed with a magnetic stirrer for 30 min at 300 rpm with the clay dispersed suspension from (b) to form a hybrid composition (c). The hybrid composition was then dried by spray drying (Büchi Mini Spray-Dryer B-290, Switzerland) using the following conditions: inlet temperature 200° C., outlet temperature 105° C., aspirator setting 100, nozzle cleaning setting 9, compressed air flow rate set at 40 mm and product flow rate 7 mL/min).

The hybrid composition was analysed by scanning electron microscopy (Zeiss Merlin High Resolution SEM (Oberkochen, Germany) located at Future Industries Institute, University of South Australia) and found to contain spherical hybrid particles of diameters in the range of 1-10 μm (as shown in FIG. 2). The inulin particles are found to encapsulate or coat the clay platelets; however, the characteristics of the hybrid composition can vary depending on the composition used and the drying method.

### Example 2—In Vivo Study

A mixture of inulin and bentonite (termed INU-BEN; 50% inulin vs 50% clay) was prepared according the Example 1.

Sprague-Dawley rats (8-week-old; OzGene, formerly Animal Resources Centre, Perth) were fed a high-fat diet (22% fat/44% energy from fat, sourced from Specialty Feeds, Glen Forrest WA) where animals were provided access to food ad libitum for a period of 3 weeks. The rats were divided into groups and administered treatments as follows: (i) PBS (negative control), (ii) INU-BEN (1 g/kg), (iii) orlistat (tetrahydrolipstatin) (positive control; 75 mg/kg; Dayang Chem (Hangzhou) Co., Ltd) and (iv) a combination group of INU-BEN (1 g/kg) and orlistat (75 mg/kg). The rats were treated daily between 3-5 μm. The experimental studies involved monitoring body weight daily, prior to dosing. Thereafter, blood samples were collected (by tail vein) prior to the rats being euthanized and were analysed for blood glucose levels and liver enzyme concentrations. The rats were dissected and analysed for fat pad weight and liver weight. The rat microbiomes were also analysed at the completion of the 3-week treatment period via faecal samples.

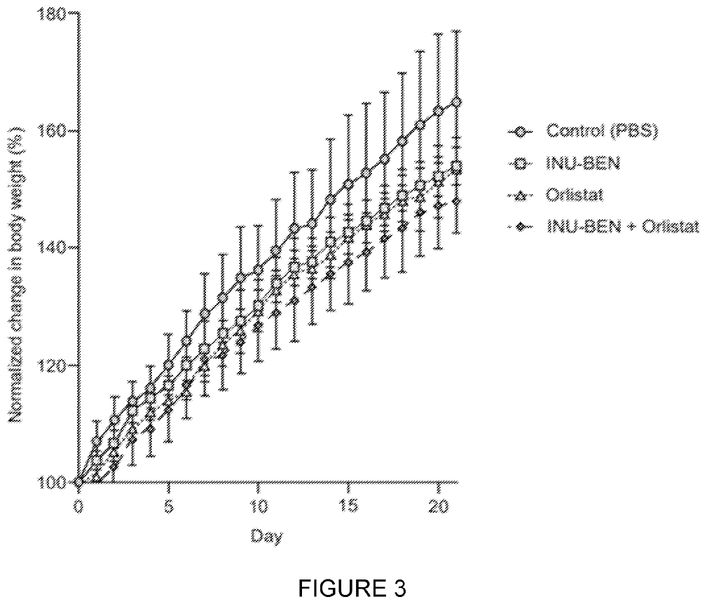

Referring to FIG. 3, the three treatment groups show a trend towards reduction in body weight gain compared to the negative (PBS) control. INU-BEN and Orlistat when administered separately performed about the same whereas the INU-BEN+Orlistat group showed the greatest reduction in body weight gain, indicating promising results.

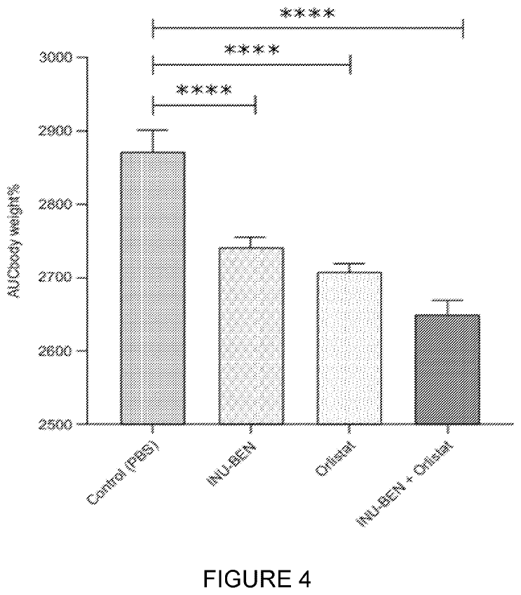

Each treatment group triggered a statistically significant reduction in body weight gain compared to the negative control as shown in FIG. 4. Referring to FIG. 4, the treatment group INU-BEN performed equivalently to that of the positive control treatment group of orlistat in reducing the area-under-the-curve, and it was also observed that there was a greater reduction in body weight of the rodents when orlistat was co-administered with INU-BEN.

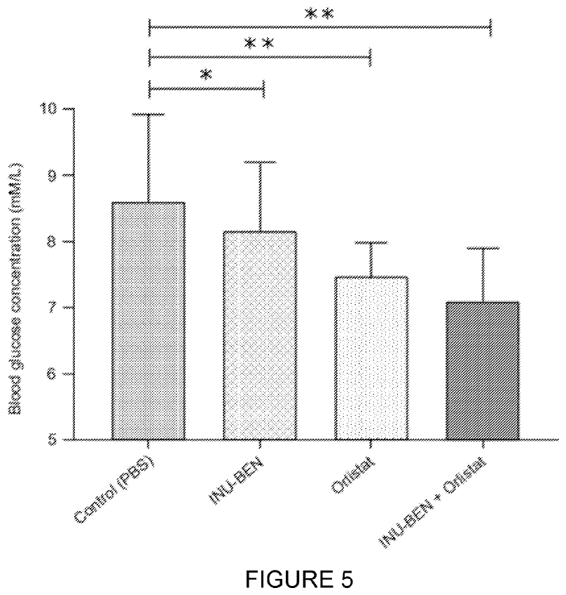

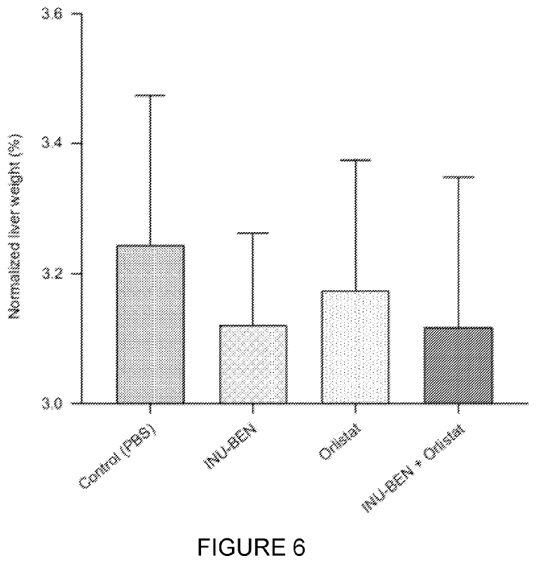

Referring to FIG. 5, plasma blood glucose concentrations were analysed following the 3-week treatment period. Each treatment group triggered a statistically significant reduction in blood glucose levels, as compared to the negative control group. Similarly, trend towards a reduction in rodent liver weight was observed for rodents treated with INU-BEN and the combination group of INU-BEN+orlistat as shown in FIG. 6. However, significance was not achieved, possibly due to the small sample size.

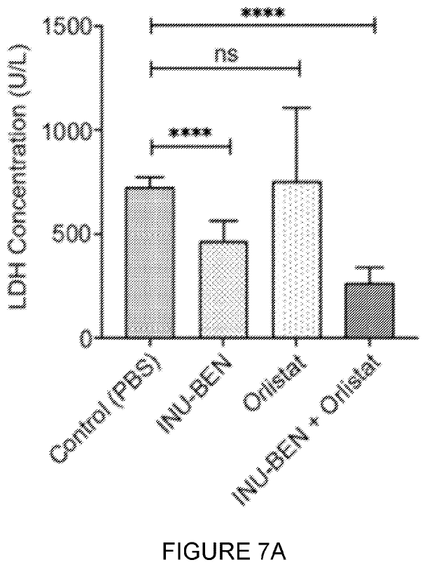

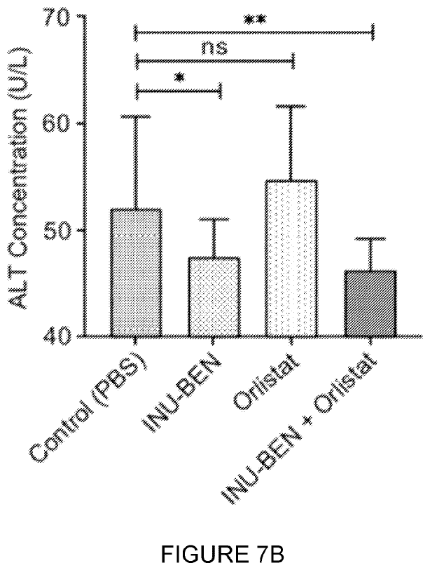

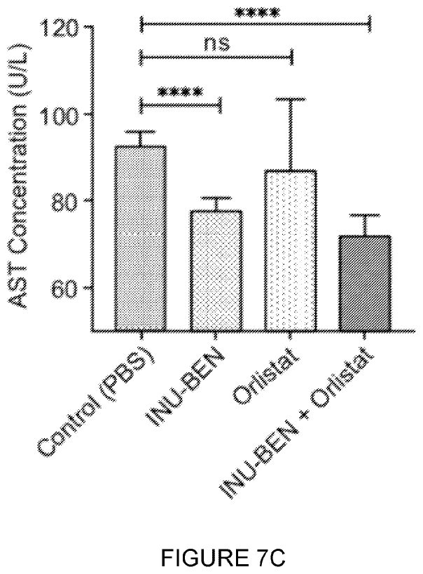

Another significant step of the experimental studies was to analyse key hepatic enzymes, Lactate dehydrogenase (LDH), Alanine aminotransferase (ALT), and aspartate aminotransferase (AST), following treatment for 3 weeks, as shown in FIGS. 7A, 7B, and 7C, respectively. The results indicated that there is an elevation in levels of each of the enzymes, thereby indicating liver damage and inflammation. Such a physiological change can be triggered by a range of health conditions, including such as, obesity. Importantly, treatment with INU-BEN and the combination group of INU-BEN+orlistat triggered a reduction in each hepatic enzyme concentration within the plasma, suggesting that each formulation can prevent liver damage and inflammation when exposed to a high-fat diet. However, dosing orlistat alone does not exert a positive impact on plasma hepatic enzyme concentrations

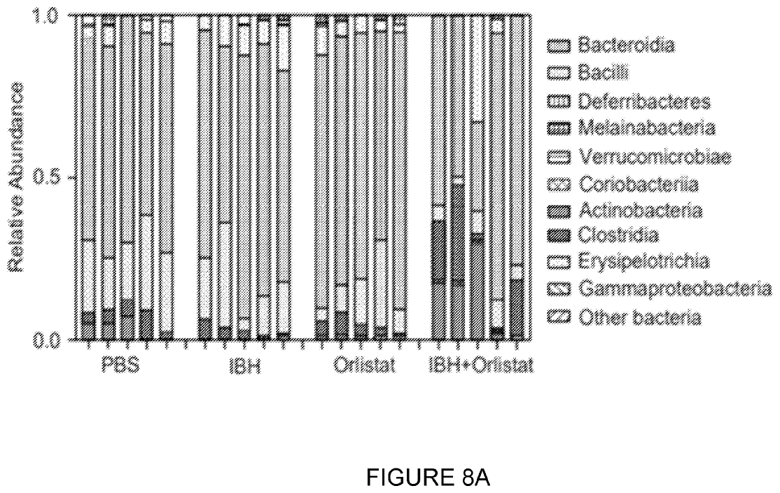

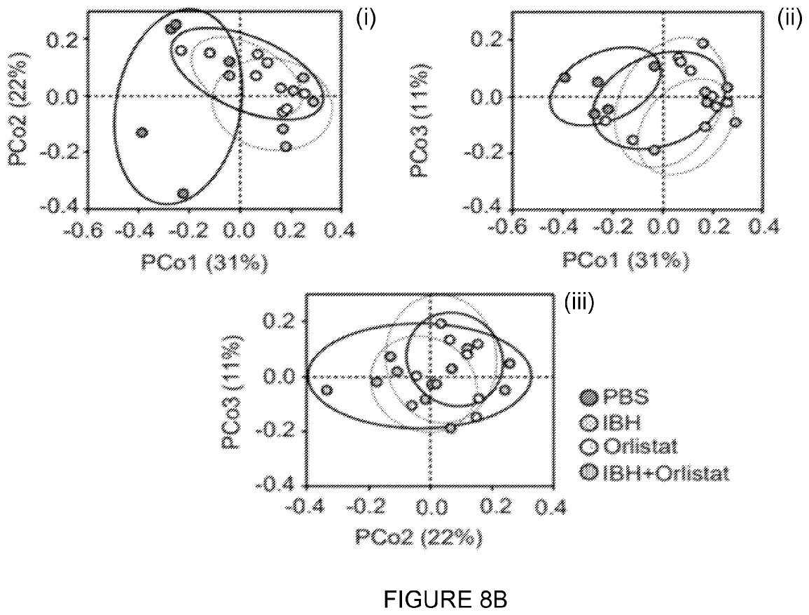

Microbiome analysis, using 16S RNA sequencing (Australian Genome Research Facility), revealed treatment-dependent changes in the relevant abundance of microbial populations, following 3 weeks of treatment is shown in a graph in FIG. 8A.

FIG. 8B illustrates a principal component analysis. This form of analysis enables the visualization of multi-dimensional data for contrasting the similarities and differences for large datasets, in this instance, microbiota data. In FIG. 8B (i), principal coordinate 1 (x axis, PCo1) is plotted against principal coordinate 2 (y axis, PCo2). Separation in PCo1 and PCo2 can be observed for the PBS group in comparison to all treatment groups, indicating that the microbiota composition is distinctly different for the PBS treated group compared to other treatment groups. In FIG. 8B (ii), PCo1 is plotted against principal coordinate 3 (PCo3) and in FIG. 8B (iii) PCo2 is plotted against PCo3. Less distinct separation in principal coordinates is evident in (ii) and (iii) with no statistically significant changes, but small shifts indicate changes to the microbiota composition. INU-BEN (IBH) groupings reveal similarities to orlistat groupings, and both were marginally separated by component 1 and component 2 compared to the control group. However, when both INU-BEN and orlistat were co-administered, there was a significant degree of separation there between. Hence, the combination treatment group leads to the most significant changes to the gut microbiome when dosed for 3 weeks.

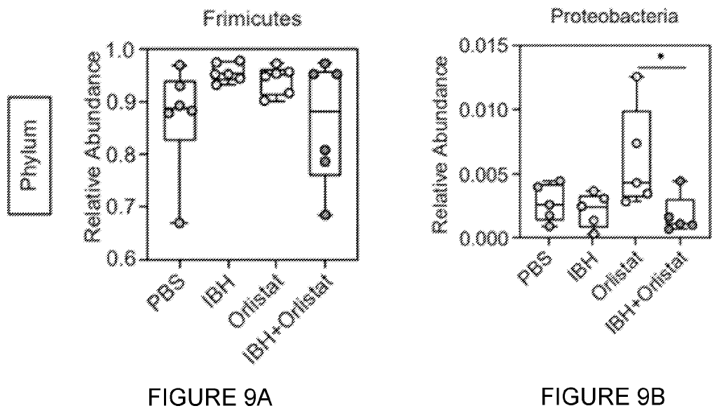

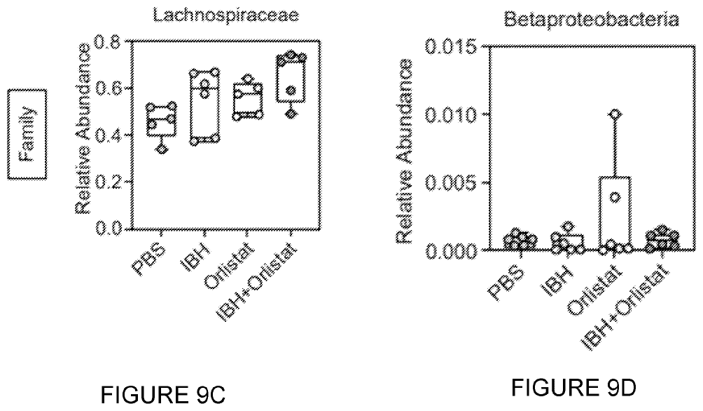

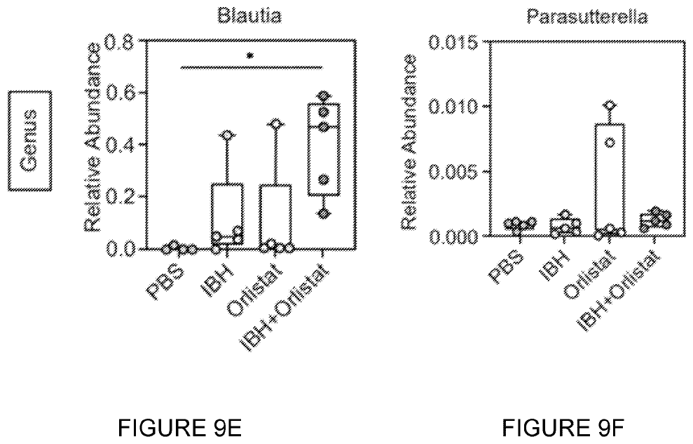

Deeper analysis relating to the relative abundance of key microbial communities demonstrated positive and negative changes induced to the gut microbiome through treatment with each group. FIGS. 9A-9F demonstrate the relative abundance of key bacteria at phylum, family and genus taxonomical levels. The bacteria Firmicutes phylum is widely considered to be beneficial for health, while the bacteria Proteobacteria phylum is associated with metabolic disorders, including obesity and irritable bowel syndrome. However, treatment of INU-BEN was shown to increase the relative abundance of health-promoting bacteria within the Firmicutes phylum as illustrated in FIG. 9A. Similar results were achieved in the case for the Blautia genus when INU-BEN was co-administered with orlistat as illustrated in FIG. 9E. A clear synergistic effect could be observed between INU-BEN and orlistat with regards to modulating the gut microbiome as shown in all the FIGS. 9A-9F. Orlistat dosing was characterized by an increase in Proteobacteria-microbial communities that are closely correlated to metabolic syndromes. Hence, orlistat not only triggers IBS-like symptoms, but also triggers negative changes to the gut microbiota. However, when orlistat was dosed with INU-BEN, the relative abundance of such harmful microbial populations was reduced, indicating that INU-BEN may prevent IBS-like changes to the gut microbiota.

Results of such studies indicated that a hybrid composition of inulin-clay particles outperformed the two precursor materials i.e., inulin and clay particles, alone. Further, results also indicated that an exemplary inulin-bentonite composition (INU-BEN) was found to have short-term anti-obesity effects in rats fed a high fat diet for a period of 3 weeks. The impact of dosing INU-BEN on the abundance of beneficial bacteria within the gut microbiome was also investigated, and it was determined that there were unexpected additive effects associated with the co-administration of INU-BEN with orlistat on key obesity phenotypes, including weight gain, blood glucose levels and liver health.

### Example 3—In Vitro Lipolysis Studies

### a) Preparation of Simulated Gastrointestinal Lipolysis Media

A two-step in vitro GI lipolysis model was employed under simulated fasted and fed state conditions to test the inhibitory effect of hybrid formulations and the precursor materials on lipase-mediated digestion in media that mimic the GI environment before (fasted state) and during (fed state) digestion of a high-fat, high-carbohydrate content meal. FaSSIF/FeSSIF/FaSSGF powder (biorelevant.com) was used to prepare fasted state simulated gastric fluid (FaSSGF; pH 1.6), fasted state simulated intestinal fluid (FaSSIF; pH 6.5), fed state simulated gastric fluid (FeSSGF; pH 5.0) and fed state simulated intestinal fluid (FeSSIF; pH 6.0). Manufacture's protocol (biorelevant.com) was followed in preparing the gastric and intestinal media, which contained biorelevant concentrations of bile salts and phospholipids. FaSSGF, FeSSGF and FaSSIF media (Biorelevant.com) were freshly prepared before each experiment and used within 48 h, according to manufacturer's recommendations. On the day of the experimental procedure, pancreatin extracts were prepared by stirring 2 g of pancreatin powder (MP Biomedicals, Seven Hills, Australia.) in 10 mL of intestinal lipolysis buffer (pH 6.5; Biorelevant.com) for 15 min, followed by centrifugation (2268 rcf, 20 min, 4° C. in a Hermle Labortechnik Model Z36HK). The supernatant was collected and kept refrigerated until use.

### b) Gastrointestinal Lipolysis Studies Under Simulated Fasted and Fed State Conditions

Details of the lipolysis media properties/excipients added to mimic the fasted and fed states are provided in Table 1. Firstly, MCT, starch and cellulose were dispersed within FaSSGF or FeSSGF (10 mL) (biorelevant.com) through continuous stirring at 600 rpm for 10 min in a thermostated glass reaction vessel (37° C.). To this dispersion, each formulation was added at 20% w/w relative to the lipid content. The pH of lipolysis medium was re-adjusted with 0.1 M NaOH or HCl to the pre-set value for FaSSGF or FeSSGF, prior to initiation of simulated gastric lipolysis via addition of lipase from Candida antarctica. Gastric lipolysis was monitored for 30 min, where the pH was continuously adjusted with 0.6 M NaOH using a pH-stat titration unit (902 Titrando, Metrohm, Switzerland) to maintain a constant pH within the digestion media. Upon completion of gastric lipolysis, FaSSIF or FeSSIF (20 mL) was added and the pH was allowed to slowly adjust via auto-titration over a 10 min period. Intestinal lipolysis was initiated through the addition of pancreatin extract and was monitored for 60 min, where the degree of free fatty acid liberation was determined by the amount of NaOH titrated to maintain a constant pH. It is important to note that NaOH titration during the gastric phase under fasted state conditions does not accurately correlate to lipolysis since fatty acids are protonated at pH 1.6. Subsequently, gastric lipolysis data is not reported.

TABLE 1
Lipolysis media compositions used
to mimic the fasted and fed state.

| Media | | | | |
|---|---|---|---|---|
| properties/ | | | | |
| excipients | Fasted state | | Fed state | |
| pH | Gastric: 1.6 | | Gastric: 5.0 | |
| | Intestinal: 6.5 | | Intestinal: 6.0 | |
| MCT (Hamilton | 200 | mg | 625 | mg |
| Laboratories, | | | | |
| Adelaide, | | | | |
| Australia) | | | | |
| Starch | — | | 625 | mg |
| (Sigma Aldrich, | | | | |
| Australia) | | | | |
| Cellulose | — | | 31 | mg |
| (Sigma Aldrich, | | | | |
| Australia) | | | | |
| Lipase | Gastric: 100 μL | | Gastric: 200 μL | |
| (Sigma Aldrich, | Candida antarctica | | Candida antarctica | |
| Australia) | (eq. to 600 TBU) | | (eq. to 1200 TBU) | |
| | Intestinal: | | Intestinal: | |
| | 2 mL pancreatin | | 2 mL pancreatin | |
| | (MP Biomedicals, Seven | | (MP Biomedicals, Seven | |
| | Hills, Australia) extract | | Hills, Australia) extract | |
| | (eq. to 2000 TBU) | | (eq. to 2000 TBU) | |
| Formulation | 40 | mg | 125 | mg |

### c) In Vitro Fermentation Studies

Inulin, bentonite and INU-BEN (50:50 ratio) formulations were prepared according to Example 1 and dosed to Blautia at varying concentrations (10 mg/mL, 50 mg/mL, 100 mg/mL) to investigate their impact on the growth of commensal gut bacteria. Blautia was plated in an anaerobic chamber at a concentration of 1×10⁵ colony forming units (CFU)/mL. Bacteria growth was measured following 24 h culture with each formulation, where growth was quantified based on the optical density of cultures at a wavelength of 600 nm.

### d) In Vivo Pharmacodynamic Studies

Four-week-old male Sprague-Dawley rats were housed in groups of two in temperature-, humidity- and pressure-controlled animal holding facility with a 12 h/12 h light/dark cycle. Following a one-week acclimatisation period, rats were placed on a high-fat diet (HFD; 22% fat/44% energy from fat) and were separated into the following treatment groups (n=6): control group (PBS; 10 mL/kg), inulin (1 g/kg), bentonite (1 g/kg) and INU-BEN (50:50 ratio) (1 g/kg) as prepared according to Example 1. Each treatment was dispersed in PBS (10 mL/kg) prior to daily evening administration between 17:00 and 19:00 via oral gavage. Evening dosing was selected for this study in an attempt to simulate rodents' natural behaviour, since they are most active and eat mostly at night. Rodent body weight was measured daily, immediately prior to dosing.

After 21 days of dosing, rats were fasted for 24 h with free access to water prior to being anaesthesized with (200 mg/kg) ilium sodium pentobarbital (60 mg/mL; Sigma Aldrich, Australia). Blood samples were collected via cardiac puncture and were sent for full blood analysis by SA Pathology (Adelaide, Australia). Following humane killing, rats were dissected and the epididymal fat pads were aseptically collected. The abundance of the pro-inflammatory cytokine, tumour necrosis factor alpha (TNF-α), was quantified in blood using enzyme-linked immunosorbent assay (ELISA) following the manufacturer's protocol (ThermoFisher).

### Results

### e) In Vitro Lipolysis Studies

In vitro lipid digestion studies were performed to assess the capacity for hybrid microparticles comprised of inulin and montmorillonite to inhibit digestion. This study serves as a suitable tool for predicting the capacity for inulin-montmorillonite hybrids to inhibit fat absorption in vivo.

Hybrid materials were dosed at 20% relative to the lipid/fat content in both the fasted and fed state. This dosing level corresponds to an approximate 5 g dose in humans, under the assumption that the average fat content of food is 25 g.

The hybrid materials and physical mix (control) of inulin and montmorillonite was at a 70:30 ratio of inulin:montmorillonite. The physical mix was prepared by combining 7 g of inulin and 3 g of montmorillonite in a glass vial and physically mixing using a spatula for 2 min prior to leaving the mixture on a rotator for overnight to ensure a homogenous mixture was formed.

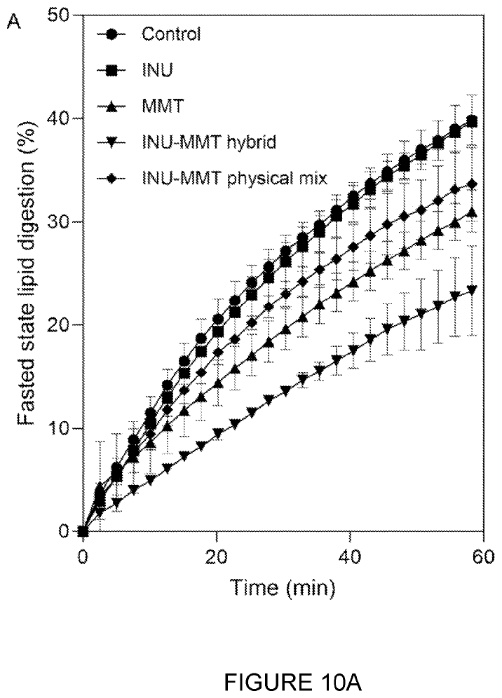

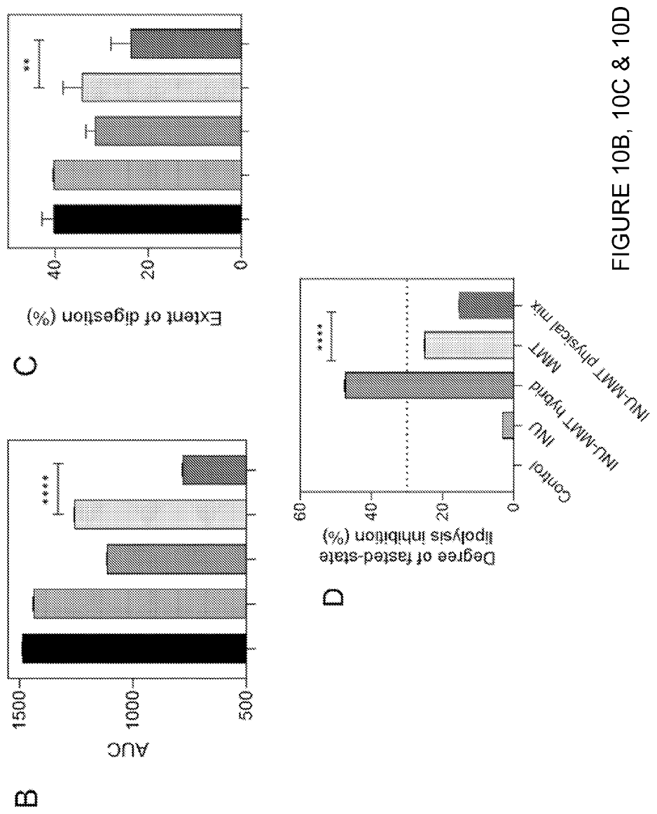

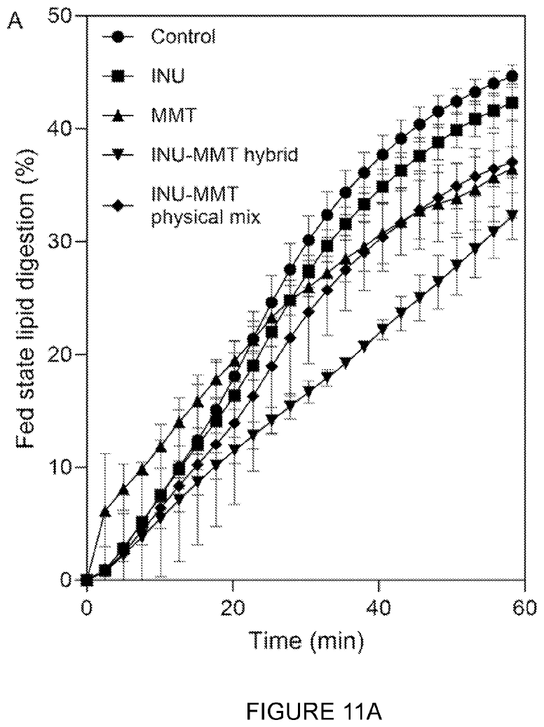

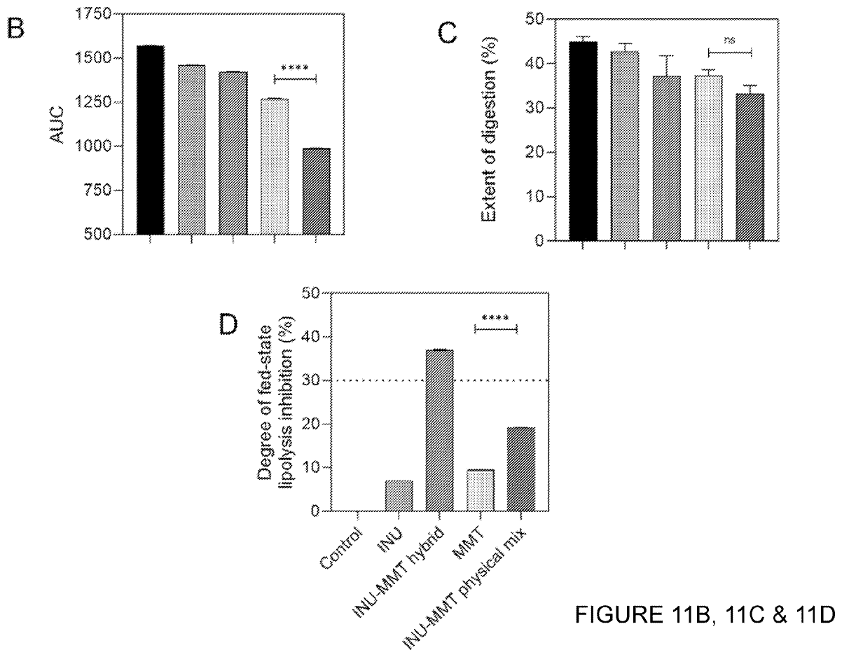

In both the fasted (FIG. 10) and fed state (FIG. 11), in vitro digestion under simulated intestinal conditions was significantly inhibited by the hybrid material when compared to both the precursor materials and the physical mix of inulin and montmorillonite, highlighting the importance of hybrid structure and composition on impeding fat digestion.

Importantly, in both the fasted and fed state, the hybrid material inhibited lipid digestion by more than 30%. This level corresponds to the desired degree of inhibition caused by orlistat (existing lipase inhibitor used for treating obesity).

### f) In Vitro Bacterial Fermentation Studies

Blautia is a genus of bacteria that is naturally present within the gut microbiota and has been shown to exert a multitude of health benefits. Thus, Blautia was selected as a model commensal bacterium for this work to investigate the impact of the developed formulations on bacteria within the gut.

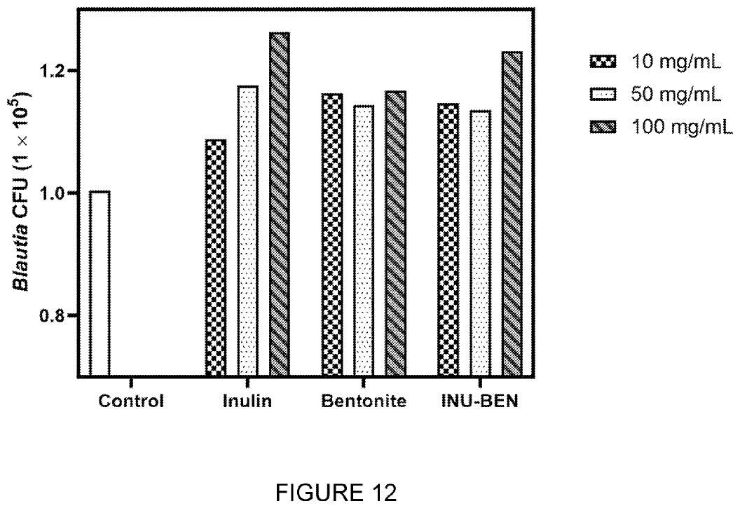

Incubating Blautia with inulin, bentonite and INU-BEN (50:50 ratio) for 24 h was shown to exert a positive impact on bacterial growth at formulation concentrations of 10, 50 and 100 mg/ml (FIG. 12).

Inulin is a well-known prebiotic fibre that is fermented by gut microbes, so the dose dependent effect on bacterial growth for inulin was expected. However, it was unclear whether bentonite would exert a positive or negative effect on bacterial growth. Thus, this study demonstrates that the bentonite present in the formulation does not impact the capacity for INU-BEN to positively modulate and promote the growth of gut bacteria.

### g) In Vivo Pharmacodynamics Study

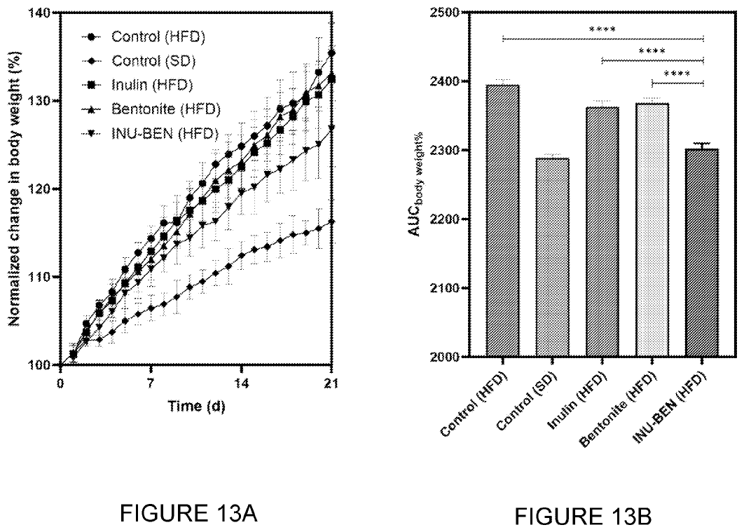

The rate and extent of body weight gain of rats exposed to a high-fat diet (HFD; 22% fat/44% energy from fat, sourced from Specialty Feeds, Glen Forrest WA) where animals were provided access to food ad libitum) was significantly reduced following daily INU-BEN administration (1 g/kg) compared to the control group (HFD) and inulin and bentonite treatment groups (FIG. 13).

The difference in area-under-the-curve (AUC) values was statistically significant for the INU-BEN group compared with the HFD control and inulin and bentonite treatment groups.

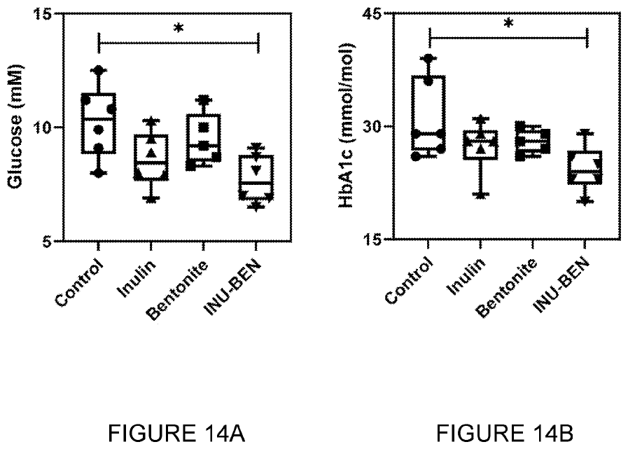

INU-BEN triggered a statistically significant reduction in blood glucose levels following 21 days of treatment, compared to the control HFD group (FIG. 14). The precursor materials did not trigger a statistically significant reduction in glucose levels, indicating that the combination of materials in a hybrid formulation is favourable for reducing glycaemic levels.

INU-BEN also triggered a statistically significant reduction in glycated haemoglobin (HbA1c) levels—a long term measure for glycaemic levels, indicating that INU-BEN successfully reduces short and long-term glycaemic levels.

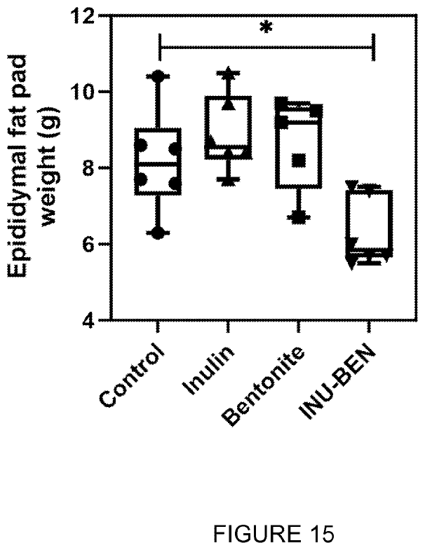

INU-BEN triggered a statistically significant reduction in epidydimal fat pad weight, suggesting that INU-BEN may potentially serve as a treatment for reducing fat stores (FIG. 15).

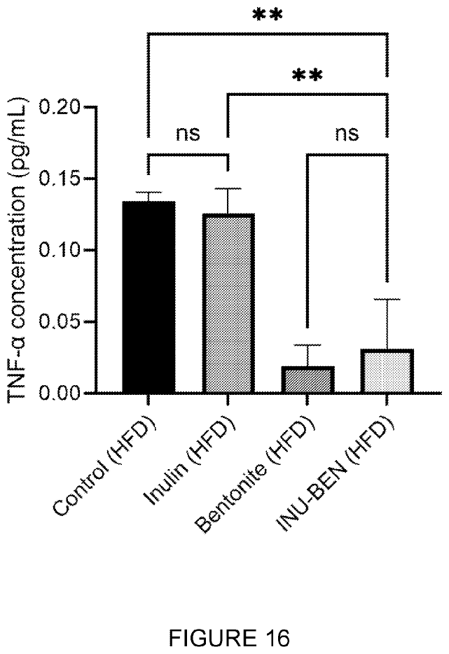

INU-BEN triggered a statistically significant reduction in plasma TNF-α concentrations, suggesting that the hybrid formulation is capable of reducing inflammation associated with obesity and metabolic syndrome (FIG. 16).

The capacity to reduce TNF-α concentrations was comparable between the bentonite and INU-BEN groups, but inulin exerted no effect on the levels of this pro-inflammatory cytokine. Importantly, this demonstrates that dosing the hybrid formulation does not compromise the capacity for bentonite to exert its anti-inflammatory response.

### Prospective Example 3—Investigating the Impact of Montmorillonite-Inulin Hybrids on Gut and Metabolic Health in Human Participants

An experiment is designed to investigate whether the supplementation of clay-inulin hybrids with a standard diet positively affects gut health and metabolic functionality.

Treatment Groups: (a) Control group; (2) Montmorillonite (1.5 g, 3 times a day; Veegum® HS; Vanderbilt Minerals, LLC); (3) Inulin (3.5 g, 3 times a day); and (4) Montmorillonite-inulin hybrids (30:70 montmorillonite:inulin ratio; eg 5 g (i.e. ~1 tsp), 3 times a day).

Rationale: A 30:70 montmorillonite:inulin ratio is selected as the preferred dose. Further, palatability and manufacturability is expected at this ratio. Additional in vitro experimentation will be performed to determine whether this ratio is effective in inhibiting fat digestion and adsorbing lipid in biorelevant conditions, specifically compared to the precursor materials (uncombined).

A high fat content ready-made meal contains approximately 25 g of fat. Thus, a 5 g dose of a montmorillonite-inulin hybrid is approximately 20% relative to the fat content in food. Several in vitro studies suggest that this ratio can provide a ≥30% reduction in fat digestion. This corresponds with the reduction in fat digestion achieved by orlistat. The in vitro studies performed on the 30:70 ratio will further confirm this.

Dosing will also be tested prior to the consumption of a meal.

Study Length: 4-12 weeks.

Rationale: Clinical studies for both inulin and montmorillonite range from 1-15 weeks in duration. Studies have shown efficacy in modulating the gut microbiome for inulin after just 1 week, however, to mitigate a study duration of at least 4-12 weeks is proposed to facilitate statistically significant changes in microbiome diversity.

Primary Measurable Outcome: Longitudinal changes in faecal microbiome composition and diversity, measured via 16S RNA sequencing.

Secondary Measurable Outcome: Metabolic and physical biomarkers related to gut health. These include but may not be limited to: glycaemic indices, blood lipid and cholesterol levels, inflammatory biomarkers, satiety biomarkers, hepatic enzyme serum concentrations, body weight, body fat percentage.

Study Population: 10-20 participants per group.

Age: 18-60 years old.

Gender: mixed.

Health status: study participants will be separated into groups of healthy population (i.e. BMI <30) and overweight/obese population (BMI >30). Successful results are expected for both groups. However, results are expected to be more pronounced in the overweight population.

Number of Participants: microbiome data is naturally variable, so a minimum of 10 people per group is preferred.

Sample Collection and Analysis: Faecal samples, blood and urine samples, and body measurement data will be collected on Day 0 prior to dosing and then weekly/fortnightly until the end of the study period, depending on study length. Final samples will be collected on the final day of the study period.

Microbial DNA will be extracted from faecal samples and sequenced using 16S RNA sequencing, performed by Australian Genome Research Facility (AGRF). SCFA metabolites will be extracted and analysed using GC-MS (performed in-house).

Full blood analysis of baseline, midpoint and endpoint samples will be performed to quantify biomarkers related to 1) metabolic health (i.e. blood triglyceride, cholesterol, leptin, glucose and HbA1c), 2) satiety (i.e. GLP-1, PYY) and 3) systemic inflammation (i.e. IL-6, IL-10, CRP). These analyses can be performed in-house.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A composition comprising:
1-99% (v/v) of an aqueous solution of at least 1% (w/v) inulin and/or one or more analogues thereof, and 1-99% (v/v) of a dispersed suspension of at least 1% (w/v) delaminated clay.

2. The composition according to claim 1, wherein the composition comprises about 30% (v/v) to about 80% (v/v) of an aqueous solution of about 1% (w/v) to about 5% (w/v) inulin and/or one or more analogues thereof, and about 30% (v/v) to about 50% (v/v) of a dispersed suspension of about 1% (w/v) to about 5% (w/v) delaminated clay.

3. A method for preparing a hybrid composition comprising 1-99% (v/v) of an aqueous solution of at least 1% (w/v) inulin and/or one or more analogues thereof, and 1-99% (v/v) of a dispersed suspension of at least 1% (w/v) clay, the method comprising the steps of:
a. preparing the aqueous solution by substantially dissolving the inulin and/or analogues thereof in a first aqueous media;
b. preparing the dispersed suspension by dispersing the clay in a second aqueous media; and
c. mixing the first aqueous solution with the second aqueous solution, to form a hybrid composition.

4. The method of claim 3, wherein the hybrid composition comprises about 1% (w/v) to about 2.5% (w/v) inulin and/or one or more analogues thereof, and about 1% (w/v) to about 2.5% (w/v) clay.

5. The method of claim 3, wherein the method comprises the step of drying the hybrid composition by spray drying.

6. The method of claim 5, wherein the dried hybrid composition comprises about 30% (w/w) to about 80% (w/w) inulin and/or one or more analogues thereof, and about 20% (w/w) to about 50% (w/w) clay.

7. (canceled)

8. (canceled)

9. A method for
(a) reducing the absorption of lipids into the bloodstream of a subject in need thereof,
(b) treating or preventing one or more metabolic disorders in a subject in need thereof, or
(c) maintaining a weight, inducing weight loss, and/or maintaining blood glucose concentrations in a subject suffering from one or more metabolic disorders,
the method comprising the step of administering to the subject a therapeutically effective amount of the composition of claim 1.

10. (canceled)

11. The composition of claim 1, wherein the inulin molecules or analogues thereof have a degree of polymerization of between 2 and 60.

12. The composition of claim 1, wherein the clay is montmorillonite and/or bentonite and/or the inulin is oligofructose.

13-16. (canceled)

17. The composition of claim 1, wherein the inulin and/or one or more analogues thereof substantially encapsulate platelets of clay forming spherical particles with diameters in the range of about 1 to about 10 μm.

18. (canceled)

19. (canceled)

20. The method claim 3, wherein the composition is subjected to a drying step and wherein the dried composition comprises (a) about 70% (w/w) inulin and/or one or more analogues thereof, and about 30% (w/w) clay, or (b) about comprises about 50% (w/w) inulin and/or one or more analogues thereof, and about 50% (w/w) clay.

21. The composition of claim 1, wherein the composition is in a unit dosage form which comprises about 1 g to about 50 g of inulin and/or one or more analogues thereof.

22. (canceled)

23. The composition of claim 1, wherein the composition is in a unit dosage form which comprises about 1 g to about 10 g of clay.

24-27. (canceled)

28. The composition of claim 1, wherein the composition is formulated for co-administration with one or more additional agents suitable for treating or preventing a metabolic disorder.

29. The composition of claim 28, wherein the one or more additional agent is selected from the group consisting of sulfonylureas, non-sulfonylurea secretagogues, insulin, insulin analogues, glucagon-like peptides, exendin-4 polypeptides, beta 3 adrenoceptor agonists, PPAR agonists, dipeptidyl peptidase IV inhibitors, biguanides, alpha-glucosidase inhibitors, immunomodulators, statins and statin-containing combinations, angiotensin converting enzyme inhibitors, adeno sine A1 receptor agonists, adenosine A2 receptor agonists, aldosterone antagonists, alpha 1 adrenoceptor antagonists, alpha 2 adrenoceptor agonists, alpha 2 adrenoceptor agonists, angiotensin receptor antagonists, antioxidants, ATPase inhibitors, atrial peptide agonists, beta adrenoceptor antagonists, calcium channel agonists, calcium channel antagonists, diuretics, dopamine D1 receptor agonists, endopeptidase inhibitors, endothelin receptor antagonists, guanylate cyclase stimulants, phosphodiesterase V inhibitors, protein kinase inhibitors, Cdc2 kinase inhibitors, renin inhibitors, thromboxane synthase inhibitors, vasopeptidase inhibitors, vasopressin I antagonists, vasopressin 2 antagonists, angiogenesis inhibitors, advanced glycation end product inhibitors, bile acid binding agents, bile acid transport inhibitors, bone formation stimulants, apolipoprotein A1 agonists, DNA topoisomerase inhibitors, cholesterol absorption inhibitors, cholesterol antagonists, cholesteryl ester transfer protein antagonists, cytokine synthesis inhibitors, DNA polymerase inhibitors, dopamine D2 receptor agonists, endothelin receptor antagonists, growth hormone antagonists, insulin sensitizers, lipase inhibitors, lipid peroxidation inhibitors, lipoprotein A antagonists, microsomal transport protein inhibitors, microsomal triglyceride transfer protein inhibitors, nitric oxide synthase inhibitors, oxidizing agents, phospholipase A2 inhibitors, radical formation agonists, platelet aggregation antagonists, prostaglandin synthase stimulants, reverse cholesterol transport activators, rho kinase inhibitors, selective estrogen receptor modulators, squalene epoxidase inhibitors, squalene synthase inhibitors, thromboxane A2 antagonists, amylin agonists, cannabinoid receptor antagonists, cholecystokinin A agonists, corticotropin-releasing factor agonists, dopamine uptake inhibitors, G protein-coupled receptor modulators, glutamate antagonists, glucagon-like peptide-1 agonists, insulin sensitizers, melanin-concentrating hormone receptor antagonists, nerve growth factor agonists, neuropeptide Y agonists, neuropeptide Y antagonists, SNRIs, protein tyrosine phosphatase inhibitors, and serotonin 2C receptor agonists.

30. The method of claim 9, wherein the metabolic disorder is selected from the group consisting of: obesity; irritable bowel syndrome (IBS); inflammatory bowel disease (IBD); diabetes mellitus; liver disease; Familial hypercholesterolemia; elevated cholesterol; Gaucher disease; Hunter syndrome; Krabbe disease; atherosclerosis; Maple syrup urine disease; hypothyroidism; Metachromatic leukodystrophy; Niemann-Pick; Phenylketonuria (PKU); Porphyria; Tay-Sachs disease; and Wilson's disease.

31. (canceled)

32. (canceled)

33. The method of claim 9, wherein the composition is administered to the subject 1-3 times per day.

34. The method of claim 9, wherein the composition is administered to the subject for at least 1-15 weeks.

35. (canceled)

36. (canceled)

37. The method of claim 9, wherein the composition is administered in the fed state or in the fasted state.

38. (canceled)

39. (canceled)
